(19) United States
(12) Patent Application Publication
(10) Pub. No.: US 2023/0242975 A1
(43) Pub. Date: Aug. 3, 2023

(54) METHODS AND SYSTEMS FOR DISTINGUISHING SOMATIC GENOMIC SEQUENCES FROM GERMLINE GENOMIC SEQUENCES

(71) Applicant: Foundation Medicine, Inc., Cambridge, MA (US)

(72) Inventors: Yanmei HUANG, Brookline, MA (US); Jason D. HUGHES, Providence, RI (US); Kyle GOWEN, Arlington, MA (US)

(73) Assignee: Foundation Medicine, Inc., Cambridge, MA (US)

(21) Appl. No.: 18/008,410

(22) PCT Filed: Jun. 3, 2021
(86) PCT No.: PCT/US2021/035751

§ 371 Date: Dec. 5, 2022

Related U.S. Application Data
(60) Provisional application No. 63/041,437, filed on Jun. 19, 2020.
(60) Provisional application No. 63/035,572, filed on Jun. 5, 2020.

(51) Int. Cl. C12Q 1/6844; C12Q 1/6886; G16B 20/20; C12Q 1/6869; G16B 30/10

(52) U.S. Cl. CPC C12Q 1/6844; C12Q 1/6886; G16B 20/20; C12Q 1/6869; G16B 30/10; C12Q 2600/16

(57) ABSTRACT

Described herein are methods for distinguishing between somatic and germline variants, and devices for implementing such methods. In certain implementation of the methods, the method can include identifying a genomic sequence of interest in a patient sample at a genomic locus; identifying one or more proxy genomic sequences for the sequence of interest; comparing an observed frequency of the sequence of interest to a centrality measure of observed frequencies of the one or more proxy genomic sequences; and based on the comparison, characterizing the genomic sequence of interest as either germline or somatic.

### CROSS-REFERENCE TO RELATED APPLICATIONS

This application claims the benefit of U.S. Provisional Patent Application No. 63/035,572, filed on Jun. 5, 2020, and of U.S. Provisional Patent Application No. 63/041,437, filed on Jun. 19, 2020, both of which are incorporated by reference herein in their entirety.

### TECHNICAL FIELD

This disclosure relates to systems and methods for distinguishing somatic genomic sequences from germline genomic sequences.

### BACKGROUND

Germline genomic sequences refer to those sequences that an organism inherits from its parents. In particular, if one or both of an organism's parents have certain genomic mutations (or if the organism experiences certain mutations in its very early development) those mutations may be germline to the organism, and will be passed to the organism's offspring (if any).

By contrast, somatic genomic sequences are sequences that are not passed from parent to child. For example, an organism may develop genomic mutations due to external factors (e.g., pollution, radiation, diet, smoking, etc.), with those genomic mutations being limited only to certain tissues, fluids, or other anatomical material. In some cases, those mutations result in undesirable medical conditions including, but not limited, to cancer.

Precision medicine is a field in which a patient is treated with a therapy that is targeted to the individual characteristics of the patient or their condition. For many patients (including cancer patients), this may involve determining genomic information about both the patient's “normal” genomic state, as well as the genomic state of the patient's “abnormal” tissue, fluid, or other anatomical material. This information may be derived from a sample from the patient, such as a tumor biopsy, a blood draw, or some other type of sample having both normal and abnormal tissue, fluid, or other anatomical material.

These samples may be assayed to determine (at least in part) the genomic sequences of the material contained therein. However, it is sometimes challenging to identify whether a particular genomic sequence comes from the patient's normal anatomical material or whether it comes from abnormal anatomical material; i.e., it is sometimes challenging to determine whether a particular genomic sequence is germline or somatic.

Understanding whether a genetic variant observed in the DNA of a cancer patient is of germline or somatic origin is critically important both in clinical practice and in cancer research. The somatic/germline distinction can be made, for example, by sequencing matched tumor and normal tissue from the same patient. Variants present in tumor but not in normal tissue are classified as somatic, whereas those present in both are classified as germline. However, such a dual-sample approach is constrained by cost as well as specimen availability. Typically in clinical practice matched normal specimens are not obtained. For example, in the case of a tissue biopsy, a single specimen containing both the tumor and its adjacent normal tissue is collected. Thus there is a need to develop methods that can reliably classify detected variants as somatic or germline in origin.

### SUMMARY

Methods, devices, and computer readable media for distinguishing somatic genomic sequences from germline genomic sequences are described herein.

Disclosed herein are methods of identifying a genomic sequence of interest as germline or somatic, the methods comprising: providing a plurality of nucleic acid molecules obtained from a sample from a subject, wherein the plurality of nucleic acid molecules comprises a mixture of tumor nucleic acid molecules and non-tumor nucleic acid molecules; optionally, ligating one or more adapters onto one or more nucleic acid molecules from the plurality of nucleic acid molecules; amplifying nucleic acid molecules from the plurality of nucleic acid molecules; capturing nucleic acid molecules from the amplified nucleic acid molecules, wherein the captured nucleic acid molecules are captured from the amplified nucleic acid molecules by hybridization to one or more bait molecules; sequencing, by a sequencer, the captured nucleic acid molecules to obtain a plurality of sequence reads corresponding to one or more genomic loci; selecting, by one or more processors, a genomic sequence of interest at a genomic locus from the one or more genomic loci; selecting, by the one or more processors, one or more proxy genomic sequences for the genomic sequence of interest; determining, by the one or more processors, an allele frequency distance using an observed allele frequency of the genomic sequence of interest and a summary statistic or distribution indicative of observed allele frequencies of the one or more proxy genomic sequences; and identifying, by the one or more processors, the genomic sequence of interest as germline or somatic using the allele frequency distance.

In some embodiments, the subject is a cancer patient. In some embodiments, the sample comprises a tissue biopsy sample, a liquid biopsy sample, a circulating tumor cell (CTC) sample, a cell-free DNA (cfDNA) sample, or a normal control. In some embodiments, the sample is a liquid biopsy sample and comprises blood, plasma, cerebrospinal fluid, sputum, stool, urine, or saliva. In some embodiments, the tumor nucleic acid molecules are derived from a tumor portion of a heterogeneous tissue biopsy sample, and the non-tumor nucleic acid molecules are derived from a normal portion of the heterogeneous tissue biopsy sample. In some embodiments, the tumor nucleic acid molecules are derived from a circulating tumor DNA (ctDNA) fraction of a cell-free DNA sample, and the non-tumor nucleic acid molecules are derived from a non-tumor fraction of the cell-free DNA sample. In some embodiments, the one or more adapters comprise amplification primers or sequencing adapters. In some embodiments, the one or more bait molecules comprise one or more nucleic acid molecules, each comprising a region that is complementary to a region of a captured nucleic acid molecule. In some embodiments, amplifying nucleic acid molecules comprises performing a polymerase chain reaction (PCR) or isothermal amplification technique. In some embodiments, the sequencing comprises use of a next generation sequencing (NGS) technique. In some embodiments, the sequencer comprises a next generation sequencer. In some embodiments, the one or more proxy genomic sequences are located within a defined segment of the subject's genomic sequence, and the selected genomic sequence of interest is located within the same defined segment. In some embodiments, the subject's genomic sequence is segmented into a plurality of segments based on copy number uniformity within each segment. In some embodiments, the summary statistic is a mean allele frequency or a median allele frequency. In some embodiments, the allele frequency distance is determined using the observed allele frequency of the genomic sequence of interest and the distribution indicative of the observed frequencies of a plurality of proxy genomic sequences, and wherein the genomic sequence of interest is identified as germline or somatic based on a probability that the observed allele frequency of the genomic sequence of interest fits within or does not fit within the distribution.

In some embodiments, a method of identifying a genomic sequence of interest as germline or somatic includes: selecting, by one or more processors, a genomic sequence of interest at a genomic locus from within a patient genomic sequence obtained for a patient sample comprising a mixture of tumor nucleic acid molecules and non-tumor nucleic acid molecules; selecting, by the one or more processors, one or more proxy genomic sequences for the genomic sequence of interest; determining, by the one or more processors, an allele frequency distance using an observed allele frequency of the genomic sequence of interest and a summary statistic or distribution indicative of observed allele frequencies of the one or more proxy genomic sequences; and identifying (e.g., classifying), by the one or more processors, the genomic sequence of interest as germline or somatic using the allele frequency distance.

In some embodiments of the method, the summary statistic is a mean allele frequency or a median allele frequency. In some embodiments, the allele frequency distance is determined using the observed allele frequency of the genomic sequence of interest and the distribution indicative of the observed frequencies of a plurality of proxy genomic sequences, and wherein the genomic sequence of interest is identified as germline or somatic based on a probability that the observed allele frequency of the genomic sequence of interest fits within or does not fit within the distribution.

In some embodiments, the tumor nucleic acid molecules and the non-tumor nucleic acid molecules comprise DNA molecules. In some embodiments, the tumor nucleic acid molecules and the non-tumor nucleic acid molecules comprise RNA molecules.

In some embodiments, the method further includes sequencing the tumor nucleic acid molecules and the non-tumor nucleic acid molecules from the patient sample to determine the patient genomic sequence. In some embodiments, the patient genomic sequence is obtained or determined using a next generation sequencing technique. In some embodiments, the sequencer is a next generation sequencer.

In some embodiments of the method, the one or more proxy genomic sequences are located within a defined segment of the patient genomic sequence, and the selected genomic sequence of interest is located within the same defined segment. In some embodiments, the patient genomic sequence is segmented into a plurality of segments based on copy number uniformity within each segment. In some embodiments, the method comprises segmenting the patient genomic sequence into a plurality of segments.

In some embodiments of the method, the patient genomic sequence is determined using targeted sequencing. In some embodiments, the targeted sequencing comprises targeted sequencing of one or more genes associated with cancer, or a portion thereof. In some embodiments, the targeted sequencing comprises targeted sequencing of one or more exon regions.

In some embodiments, the method includes: identifying, by one or more processors, a genomic sequence of interest in a patient sample at a genomic locus; identifying, by the one or more processors, one or more proxy genomic sequences for the sequence of interest; comparing, by the one or more processors, an observed frequency of the sequence of interest to a centrality measure of observed frequencies of the one or more proxy genomic sequences; and based on the comparison, identifying (e.g., classifying or characterizing) the genomic sequence of interest as either germline or somatic.

In some embodiments of the method, the one or more proxy genomic sequences includes a single nucleotide polymorphism (SNP).

In some embodiments of the method, the one or more proxy genomic sequences includes an allele.

In some embodiments, the method further comprises identifying, by the one or more processors, a segment of a patient's genome in which the genomic locus is included. In some embodiments, identifying, by the one or more processors, the segment includes performing a segmentation procedure on a continuous portion of the patient's genome. In some embodiments, the portion of the patient's genome is large enough to identify three distinct segments. In some embodiments, the proxy is identified, by the one or more processors, to be located on the same segment as the genomic locus. In some embodiments, the segmentation procedure identifies segments according to whether a genomic parameter is equal across the entirety of each individual segment. In some embodiments, the genomic parameter is copy number.

In some embodiments of any of the above methods of identifying a genomic sequence of interest as germline or somatic, the step of identifying, by the one or more processors, the genomic sequence of interest as germline or somatic includes: inputting an allele frequency distance into a trained statistical model; and outputting, from the trained statistical model, a value indicative of a likelihood that the genomic sequence of interest is germline or a value indicative of a likelihood that the genomic sequence of interest is somatic. In some embodiments, the allele frequency distance is adjusted to correct for a contamination level in the patient sample, a low sequencing read depth, a noisy estimation of allele frequencies, a low segment germline single nucleotide polymorphism (SNP) count, or high variability in segment germline SNP allele frequency. In some embodiments, the trained statistical model comprises a function that associates the allele frequency distance with the value indicative of a likelihood that the genomic sequence of interest is germline or the value indicative of a likelihood that the genomic sequence of interest is somatic.

In some embodiments, the trained statistical model is a logistic regression model. In some embodiments, the trained statistical model is trained using tumor samples with known germline sequences. In some embodiments, the trained statistical model is trained using data for tumor samples with known germline sequences and known somatic sequences. In some embodiments, the method further comprises training the statistical model using data for tumor samples with known germline sequences. In some embodiments, the method further comprises training the statistical model using data for tumor samples with known germline sequences and known somatic sequences.

In some embodiments, the trained statistical model is trained using data for variant allele frequencies that excludes variants located in genomic regions known to have allele frequencies that deviate from expected values. In some embodiments, the method further comprises training the statistical model using data for variant allele frequencies that excludes variants located in genomic regions known to have allele frequencies that deviate from expected values.

In some embodiments, the trained statistical model is trained using data that incorporates prior knowledge of the likelihood of a variant being a germline, a somatic variant, or a clonal hematopoiesis of indeterminate potential (CHIP) variant based on historical data or databases. In some embodiments, the method further comprises training the statistical model using data that incorporated prior knowledge of the likelihood of a variant being a germline, a somatic variant, or a clonal hematopoiesis of indeterminate potential (CHIP) variant based on historical data or databases.

In some embodiments, the trained statistical model is trained using data that accounts for a noise level for a given variant call and its genomic context. In some embodiments, the method further comprises training the statistical model using data that accounts for a noise level for a given variant call and its genomic context.

In some embodiments, the one or more proxy genomic sequences include a single nucleotide polymorphism (SNP). In some embodiments, the one or more proxy genomic sequences include an allele. In some embodiments of the method, the genomic sequence of interest includes a genomic variant.

In some embodiments of the method, the method further comprises generating, by the one or more processors, a report indicating the genomic sequence of interest as either germline or somatic. In some embodiments, the method comprises transmitting the report, for example to a healthcare provider. In some embodiments, the report is transmitted via a computer network or a peer-to-peer connection.

In some embodiments of any of the above methods, the patient sample is derived from a tissue biopsy comprising tumor tissue and non-tumor tissue. In some embodiments, the tissue biopsy is a solid tissue biopsy or a liquid biopsy. In some embodiments, the tissue biopsy is a liquid biopsy comprising blood, plasma, cerebrospinal fluid, sputum, stool, urine, or saliva. In some embodiments, the patient sample comprises cell-free DNA (cdDNA) obtained from the subject. In some embodiments, the patient sample comprises circulating tumor DNA (ctDNA) obtained from the subject.

Further described herein is a method of treating cancer in a patient, which includes identifying, by the one or more processors, one or more genomic sequences of interest as somatic using any of the methods described above; selecting a cancer treatment modality based on the one or more identified somatic sequences; and treating the cancer using the selected cancer treatment modality. In some embodiments, the one or more identified somatic sequences are associated with successful cancer treatment using the selected treatment modality. In some embodiments, the method comprises determining, by the one or more processors, a microsatellite instability status of the cancer using the one or more identified somatic sequences; and selecting the cancer treatment modality based on the microsatellite instability status of the cancer. In some embodiments, the method includes determining, by the one or more processors, a tumor mutational burden for the cancer using the one or more identified somatic sequences; and selecting the cancer treatment modality based on the tumor mutational burden being above a predetermined tumor mutational burden threshold. In some embodiments, the cancer treatment modality comprises administration of an effective amount of one or more anti-cancer agents to the patient if the tumor mutational burden is above a predetermined threshold. In some embodiments, the one or more anti-cancer agents comprises an immuno-oncology agent. In some embodiments, the immuno-oncology agent is an immune checkpoint inhibitor.

Also described herein is a method of monitoring cancer progression or recurrence in a patient, which includes identifying, by the one or more processors, one or more genomic sequences of interest as somatic using any of the methods described above; and detecting, by the one or more processors, the presence or absence of the one or more genomic sequences of interest identified as somatic within a second patient sample obtained from patient after the cancer has been treated. In some embodiments, the method comprises obtaining the second patient sample from the patient. In some embodiments, the method comprises treating the cancer in the patient after the first patient sample is obtained from the patient and before the second patient sample is obtained from the patient. In some embodiments, the second patient sample comprises cell-free DNA. In some embodiments, detecting the presence or absence of the one or more genomic sequences of interest identified as somatic within the second patient sample comprises sequencing nucleic acid molecules in the second patient sample.

Further described herein is a method of selecting a neoantigen for a cancer vaccine personalized for a subject having cancer, comprising: identifying, by the one or more processors, one or more genomic sequences of interest as somatic using any of the methods described above, wherein the one or more genomic sequences of interest identified as somatic is located within an exon region of a gene; and selecting, by the one or more processors, from the one or more genomic sequences of interest identified as somatic, a genomic sequence that encodes a neoantigen suitable as a cancer vaccine for the subject. In some embodiments, the method comprises making a vaccine comprising the neoantigen.

Also described herein is a non-transitory computer-readable storage medium storing one or more programs, the one or more programs comprising instructions, which when executed by one or more processors of an electronic device, cause the electronic device to: select a genomic sequence of interest at a genomic locus from within a patient genomic sequence obtained for a patient sample comprising a mixture of tumor nucleic acid molecules and non-tumor nucleic acid molecules; select one or more proxy genomic sequences for the genomic sequence of interest; determine an allele frequency distance using an observed allele frequency of the genomic sequence of interest and a summary statistic or distribution indicative of observed allele frequencies of the one or more proxy genomic sequences; and identify the genomic sequence of interest as germline or somatic using the allele frequency distance. In some embodiments, the summary statistic is a mean allele frequency or a median allele frequency. In some embodiments, the allele frequency distance is determined using the observed allele frequency of the genomic sequence of interest and the distribution indicative of the observed frequencies of a plurality of proxy genomic sequences, and wherein the genomic sequence of interest is identified as germline or somatic based on a probability that the observed allele frequency of the genomic sequence of interest fits within or does not fit within the distribution. In some embodiments, the tumor nucleic acid molecules and the non-tumor nucleic acid molecules comprise DNA molecules. In some embodiments, the tumor nucleic acid molecules and the non-tumor nucleic acid molecules comprise RNA molecules.

In some embodiments of the non-transitory computer-readable storage medium, the one or more proxy genomic sequences are located within a defined segment of the patient genomic sequence, and the selected genomic sequence of interest is located within the same defined segment. In some embodiments, the patient genomic sequence is segmented into a plurality of segments based on copy number uniformity within each segment.

In some embodiments of the non-transitory computer-readable storage medium, the one or more programs further comprise instructions, which when executed by the one or more processors of the electronic device, cause the electronic device to segment the patient genomic sequence into a plurality of segments.

In some embodiments of the non-transitory computer-readable storage medium, the patient genomic sequence is determined using targeted sequencing. In some embodiments, the patient genomic sequence is determined using next generation sequencing. In some embodiments, the targeted sequencing comprises targeted sequencing of one or more genes associated with cancer, or a portion thereof. In some embodiments, the targeted sequencing comprises targeted sequencing of one or more exon regions.

In some embodiments, a non-transitory computer-readable storage medium stores one or more programs, the one or more programs comprising instructions, which when executed by one or more processors of an electronic device, cause the electronic device to: identify a genomic sequence of interest in a patient sample at a genomic locus; identify one or more proxy genomic sequences for the sequence of interest; identify an observed frequency of the sequence of interest to a centrality measure of observed frequencies of the one or more proxy genomic sequences; and based on the comparison, characterize the genomic sequence of interest as either germline or somatic.

In some embodiments of the non-transitory computer readable storage medium, the one or more programs further comprise instructions, which when executed by the one or more processors of the electronic device, cause the electronic device to generate a report indicating the genomic sequence of interest as either germline or somatic. In some embodiments, the electronic device comprises a display, and the one or more programs further comprise instructions, which when executed by the one or more processors of the electronic device, cause the electronic device to display the report.

In some embodiments of the non-transitory computer readable storage medium, the one or more proxy genomic sequences includes a single nucleotide polymorphism (SNP).

In some embodiments of the non-transitory computer readable storage medium, the one or more proxy genomic sequences includes an allele.

In some embodiments of the non-transitory computer readable storage medium, the one or more programs further comprise instructions, which when executed by the one or more processors of the electronic device, cause the electronic device to identify a segment of a patient's genome in which the genomic locus is included. In some embodiments, identifying the segment includes performing a segmentation procedure on a continuous portion of the patient's genome. In some embodiments, the portion of the patient's genome is large enough to identify three distinct segments. In some embodiments, the one or more proxy genomic sequence are identified to be located on the same segment as the genomic locus. In some embodiments, the segmentation procedure identifies segments according to whether a genomic parameter is equal across the entirety of each individual segment. In some embodiments, the genomic parameter is copy number.

In some embodiments of the non-transitory computer readable storage medium, the genomic sequence of interest includes a genomic variant.

In some embodiments of the non-transitory computer readable storage medium, the one or more programs further comprise instructions, which when executed by one or more processors of the electronic device, cause the electronic device to receive sequencing data associated with the patient genomic sequence. In some embodiments, the one or more programs further comprise instructions, which when executed by one or more processors of the electronic device, cause the electronic device to assemble the patient genomic sequence using the sequencing data. In some embodiments, the one or more programs further comprise instructions, which when executed by one or more processors of the electronic device, cause the electronic device to operate a sequencer to sequence nucleic acid molecules derived from the patient sample, thereby obtaining the sequencing data.

In some embodiments of the non-transitory computer readable storage medium, the one or more programs further comprise instructions, which when executed by the one or more processors of the electronic device, cause the electronic device to generate a report indicating the genomic sequence of interest as either germline or somatic. In some embodiments, the one or more programs further comprise instructions, which when executed by the one or more processors of the electronic device, cause the electronic device to transmit the report using a computer network.

In some embodiments of the non-transitory computer readable storage medium, the electronic device comprises a display, and the one or more programs further comprise instructions, which when executed by the one or more processors of the electronic device, cause the electronic device to display the report.

In some embodiments of the non-transitory computer readable storage medium, the one or more proxy genomic sequences includes a single nucleotide polymorphism (SNP).

In some embodiments of the non-transitory computer readable storage medium, the one or more proxy genomic sequences includes an allele.

In some embodiments of the non-transitory computer readable storage medium, the genomic sequence of interest includes a genomic variant.

Also described herein is an electronic device, comprising: one or more processors; and a memory storing one or more programs configured to be executed by the one or more processors, the one or more programs including instructions for: selecting a genomic sequence of interest at a genomic locus from within a patient genomic sequence obtained for a patient sample comprising a mixture of tumor nucleic acid molecules and non-tumor nucleic acid molecules; selecting one or more proxy genomic sequences for the genomic sequence of interest; determining an allele frequency distance using an observed allele frequency of the genomic sequence of interest and a summary statistic or distribution indicative of observed allele frequencies of the one or more proxy genomic sequences; and identifying the genomic sequence of interest as germline or somatic using the allele frequency distance. In some embodiments, the summary statistic is a mean allele frequency or a median allele frequency. In some embodiments, the allele frequency distance is determined using the observed allele frequency of the genomic sequence of interest and the distribution indicative of the observed frequencies of a plurality of proxy genomic sequences, and wherein the genomic sequence of interest is identified as germline or somatic based on a probability that the observed allele frequency of the genomic sequence of interest fits within or does not fit within the distribution. In some embodiments, the tumor nucleic acid molecules and the non-tumor nucleic acid molecules comprise DNA molecules. In some embodiments, the tumor nucleic acid molecules and the non-tumor nucleic acid molecules comprise RNA molecules. In some embodiments, the patient genomic sequence is determined using next generation sequencing.

In some embodiments of the electronic device, the one or more proxy genomic sequences are located within a defined segment of the patient genomic sequence, and the selected genomic sequence of interest is located within the same defined segment. In some embodiments, the patient genomic sequence is segmented into a plurality of segments based on copy number uniformity within each segment. In some embodiments, the one or more programs further include instructions for segmenting the patient genomic sequence into a plurality of segments.

In some embodiments of the electronic device, the patient genomic sequence is determined using targeted sequencing. In some embodiments, the targeted sequencing comprises targeted sequencing of one or more genes associated with cancer, or a portion thereof. In some embodiments, the targeted sequencing comprises targeted sequencing of one or more exon regions.

In some embodiments, an electronic device, comprises: one or more processors; and a memory storing one or more programs configured to be executed by the one or more processors, the one or more programs including instructions for: identifying a genomic sequence of interest in a patient sample at a genomic locus; identifying one or more proxy genomic sequences for the sequence of interest; comparing an observed frequency of the sequence of interest to a centrality measure of observed frequencies of the one or more proxy genomic sequences; and based on the comparison, characterizing the genomic sequence of interest as either germline or somatic.

In some embodiments of the electronic device, the one or more proxy genomic sequences includes a single nucleotide polymorphism (SNP).

In some embodiments of the electronic device, the one or more proxy genomic sequences includes an allele.

In some embodiments of the electronic device, the one or more programs further include instructions for identifying a segment of a patient's genome in which the genomic locus is included. In some embodiments, identifying the segment includes performing a segmentation procedure on a continuous portion of the patient's genome. In some embodiments, the portion of the patient's genome is large enough to identify three distinct segments. In some embodiments, the proxy is identified to be located on the same segment as the genomic locus. In some embodiments, the segmentation procedure identifies segments according to whether a genomic parameter is equal across the entirety of each individual segment. In some embodiments, the genomic parameter is copy number.

In some embodiments of the electronic device, the genomic sequence of interest includes a genomic variant.

In some embodiments of the electronic device, the one or more programs further comprise instructions for receiving sequencing data associated with the patient genomic sequence. In some embodiments, the one or more programs further comprise instructions for assembling the patient genomic sequence using the sequencing data. In some embodiments, the one or more programs further comprise instructions for causing a sequencer to sequence nucleic acid molecules derived from the patient sample, thereby obtaining the sequencing data.

In some embodiments of the electronic device, the one or more proxy genomic sequences includes a single nucleotide polymorphism (SNP).

In some embodiments of the electronic device, the one or more proxy genomic sequences includes an allele.

In some embodiments of the electronic device, the genomic sequence of interest includes a genomic variant.

In some embodiments of the electronic device, the one or more programs further include instructions for generating a report indicating the genomic sequence of interest as either germline or somatic. In some embodiments, the one or more programs further include instructions for transmitting the report via a computer network or a peer-to-peer connection. In some embodiments, the device further comprises a display and the one or more programs further include instructions for displaying the report.

In some embodiments of the electronic device, the patient sample is derived from a tissue biopsy comprising tumor tissue and non-tumor tissue. In some embodiments, the tissue biopsy is a solid tissue biopsy or a liquid biopsy. In some instances, the tissue sample is a liquid biopsy comprising blood, plasma, cerebrospinal fluid, sputum, stool, urine, or saliva. In some embodiments, the patient sample comprises cell-free DNA (cfDNA) obtained from the subject. In some embodiments, the patient sample comprises circulating tumor DNA (ctDNA) obtained from the subject.

Also described herein is a system, comprising any of the electronic devices described herein and a sequencer configured to sequence nucleic acid molecules derived from the patient sample. In some embodiments, the sequencer is a next generation sequencer.

Disclosed herein are methods of identifying a genomic sequence of interest as germline or somatic, the methods comprising: identifying, by one or more processors, a genomic sequence of interest in a patient sample at a genomic locus; identifying, by the one or more processors, a proxy genomic sequence for the genomic sequence of interest; comparing, by the one or more processors, an observed allele fraction of the genomic sequence of interest to an observed allele fraction of the proxy genomic sequence; and identifying, by the one or more processors, the genomic sequence of interest as germline or somatic based on the comparison. In some embodiments, the proxy genomic sequence has the same copy number as the genomic sequence of interest. In some embodiments, identifying, by the one or more processors, the genomic sequence of interest as germline or somatic comprises: inputting an allele frequency distance into a trained statistical model; and outputting, from the trained statistical model, a value indicative of a likelihood that the genomic sequence of interest is germline or a value indicative of a likelihood that the genomic sequence of interest is somatic. In some embodiments, the allele fraction of the genomic sequence and the allele fraction of the proxy genomic sequence are determined using a next generation sequencing technique. In some embodiments, the allele fraction of the genomic sequence and the allele fraction of the proxy genomic sequence are determined using a microarray technique. In some embodiments, the patient sample comprises a solid tissue biopsy or a liquid biopsy. In some embodiments, the patient sample is a liquid biopsy comprising blood, plasma, cerebrospinal fluid, sputum, stool, urine, or saliva. In some embodiments, the patient sample comprises cell-free DNA (cfDNA) obtained from the subject. In some embodiments, the patient sample comprises circulating tumor DNA (ctDNA) obtained from the subject. In some embodiments, the patient is a cancer patient.

### INCORPORATION BY REFERENCE

All publications, patents, and patent applications mentioned in this specification are herein incorporated by reference in their entirety to the same extent as if each individual publication, patent, or patent application was specifically and individually indicated to be incorporated by reference in its entirety. In the event of a conflict between a term herein and a term in an incorporated reference, the term herein controls.

### DESCRIPTION OF DRAWINGS

FIG. 1 is a schematic depiction of a section of a patient's genome.

FIG. 2 is a flowchart for a process to distinguish germline and somatic genomic sequences.

FIG. 3 is a schematic depiction of genomic segmentation.

FIG. 4 illustrates an exemplary system including an electronic device, which may be used to execute the methods described herein.

FIG. 5A shows an exemplary process for determining the difference in expected variant allele fraction for somatic and germline variants given the same tumor fraction, ploidy, and copy number.

FIG. 5B shows an exemplary method for determining the allele frequency distance from expected germline allele frequency (AFDIS), and an exemplary density distribution of AFDIS, from which an empirical cumulative distribution function (ECDF) can be built.

FIG. 5C shows an exemplary plot of AFDIS, plotted against the computed purity of the tumor samples.

FIG. 5D shows a non-limiting example of an ROC curve for classification of somatic and germline variants in tumor samples according to a method disclosed herein.

FIG. 5E shows a non-limiting example of probability plot for an exemplary logistic regression model that may be used with some embodiments.

FIG. 5F shows a plot of the somatic probability of different variants determined using an exemplary logistic regression model.

FIG. 5G shows the improvement of the claimed methods over a prior SGZ method.

FIG. 5H shows a non-limiting example of a sensitivity plot for the training data and test data used to train and test a logistic regression model according to an exemplary method disclosed herein.

FIG. 5I shows a non-limiting example of a positive predictive value (PPV) plot for the training data and test data used to train and test a logistic regression model according to an exemplary method disclosed herein.

FIG. 5J shows a non-limiting example of data for the classification of variants in the BRCA1 and BRCA2 genes using an exemplary embodiment of the described methods.

FIG. 5K shows a non-limiting example of data for the classification of variants in the STH11 gene using an exemplary embodiment of the described methods.

FIG. 6A shows a non-limiting example of a plot of variant allele frequency (AF) versus segment minor allele frequency (MAF) for known germline variants in tumor samples.

FIG. 6B shows non-limiting examples of density versus variant AF plots corresponding to segment MAF values of 0.1, 0.2, and 0.3, respectively, as derived from the data plotted in FIG. 6A.

### DETAILED DESCRIPTION

Methods, devices, and computer readable media for distinguishing somatic genomic sequences from germline genomic sequences are described herein. A genomic sequence of interest in a patient sample at a genomic locus can be identified. Then, for the sequence of interest, one or more proxy genomic sequences can be identified. The observed frequency of the sequence of interest can be compared to a centrality measure of observed frequencies of the one or more proxy genomic sequences; and, based on the comparison, the genomic sequence of interest can be characterized as either a germline sequence or a somatic sequence.

Several methods had been developed in the past to determine somatic/germline status of variants in a single-sample setting, including matching to public germline databases such as dbSNP, or using surrogates constructed from a large number of normal individuals in place of the matched normal. See, for example, Hiltemann, et al., Discriminating somatic and germline mutations in tumor DNA samples without matching normal, Genome Res. vol. 25, no. 9, pp. 1382-1390 (2015). However, such methods are ineffective when dealing with rare germline variants that are limited to a family or small population. There is also a so called “basic method”, in which variants with allele frequency (or allele fraction) near 50% or 100% are regarded as germline and those not satisfying this criterion are classified as somatic. See Jones, et al., Personalized genomic analyses for cancer mutation discovery and interpretation, Sci. Transl. Med., vol. 7, no. 283, p. 283ra53 (2015). This basic method fails to account for the fact that aneuploidy can drive allele frequency of germline variants significantly away from the 50% or 100% expectations. The terms “allele frequency” and “allele fraction” are used interchangeably herein and refer to the fraction of sequence reads corresponding to a particular allele relative to the total number of sequence reads for a genomic locus.

Published in early 2018, the SGZ (somatic-germline-zygosity) algorithm sought to provide a solution to the single-sample somatic/germline classification problem by accounting for tumor content, tumor ploidy, and the local copy number. SGZ was demonstrated to greatly out-perform the “basic method” in somatic/germline calling accuracy in validation datasets (Sun, et al., A computational approach to distinguish somatic vs. germline origin of genomic alterations from deep sequencing of cancer specimens without a matched normal, PLoS Comput Biol., vol. 14, no. 2, p. e1005965 (2018), which is incorporated herein by reference in its entirety). Application of the SGZ algorithm in FMI's deep massively parallel sequencing (MPS)-based diagnostic products has enabled effective somatic/germline status determination for short variants (substitutions and indels) and became an indispensable tool for applications such as Tumor Mutational Burden (TMB) estimation.

The methods described herein for somatic/germline classification represent a further improvement over the SGZ approach. The new approach is built upon the same underlying principle, i.e. in a tumor/normal admixture, somatic and germline variants often have different expected allele frequencies that are dictated by tumor fraction, tumor ploidy and local copy number. However, in contrast to SGZ, which estimates expected germline allele frequency by computational modeling of tumor fraction, tumor ploidy and local copy number, the new methods disclosed herein directly infer the expected germline allele frequency from known germline SNPs located on the same copy number segment with the variant in question. Thus, using the method described herein, it is not necessary to determine or model the copy number or tumor purity to obtain an accurate call for somatic and germline variants.

In some embodiments, a trained model, such as a logistic regression model, is used to predict probability of a variant being somatic based on the difference between the observed variant allele frequency and the inferred expected germline variant allele frequency. In some embodiments, the model is trained using data for matched tumor/normal pairs and validated with independent datasets. In some embodiments, the model is trained using data for tumor samples with known germline (and, optionally, known somatic) sequences. In some embodiments, the model is trained using data for mixed tumor/normal samples with known germline (and, optionally, known somatic) sequences. The validation shows the new classifier outperforms SGZ in sensitivity and positive predictive value (PPV) for somatic variant classification.

A determined genomic sequence may be a somatic variant sequence or a germline sequence. Publicly accessible databases of known germline sequences exist (see, for example, dbSNP (available at www.ncbi.nlm.nih.gov/snp/) or gnomAD (available at gnomad.broadinstitute.org)), and a match between a known germline sequence and a sequence determined by sequencing nucleic acids in a sample obtained from a subject indicates that the sequence associated with the sample is likely to be a germline sequence. However, failure to match a known germline sequence does not demonstrate that the sequence is a somatic variant sequence, as it could be a previously unknown (or unrecorded) germline sequence of the subject. The methods described herein allow for the classification of the sequence as a germline sequence or somatic variant sequence.

### Methods for Calling Somatic or Germline Sequences

Methods described herein allow for the identification of a genomic sequence of interest as a germline sequence or a somatic sequence. In some embodiments, the somatic sequence is associated with a cancer in a patient. For example, a patient sample can include a mixture of tumor nucleic acid molecules (i.e., nucleic acid molecules derived from a tumor, either directly (such as in the case of a tumor biopsy) or indirectly (such as in the case of a liquid biopsy or bodily fluid sample comprising circulating-tumor DNA (ctDNA) as well as cell-free DNA (cfDNA)) and non-tumor nucleic acid molecules (i.e., nucleic acid molecules derived from non-tumorous, and preferably healthy, tissue, cells, liquid biopsy samples, or bodily fluid samples). The methods may include a step of selecting a genomic sequence of interest from within a patient genomic sequence (i.e., a genomic sequence obtained for the patient, which may be a whole genome or a portion thereof (e.g., an exome or a targeted region within the whole genome)), and a step of selecting one or more proxy genomic sequences for the genomic sequence of interest. The patient genomic sequence may include one or more alleles at any given locus (e.g., a somatic sequence and/or a germline sequence at any given locus).

Nucleic acid molecules from a sample (for example, a mixed tumor/normal tissue sample, or a cell-free DNA (cfDNA) sample containing a mixture of ctDNA and non-tumor cfDNA) can be sequenced to determine a patient genomic sequence. A genomic sequence of interest can be identified or selected at a genomic locus from the patient genomic sequence. The selected genomic sequence is a test sequence which is to be characterized as germline or somatic. In some embodiments, the genomic sequence of interest differs from a reference sequence. In some embodiments, the genomic sequence of interest differs from a sequence in a selected germline sequence database.

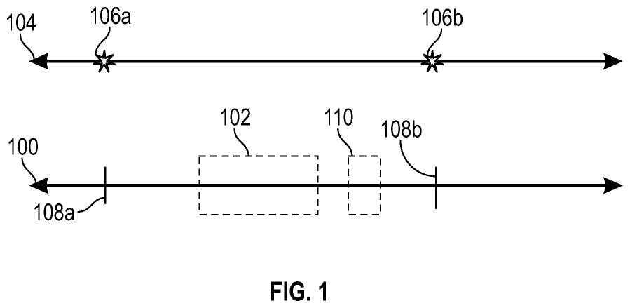

FIG. 1 is a schematic depiction of a sample genomic region. The region 100 may include the entire genome of an organism, or may include only a fraction of the entire genome. Although the region 100 is shown as a continuous line in FIG. 1, in general the region 100 may include several components that are physically separated on the organism's chromosome(s). In some implementations, the sample from which the region 100 is determined may include normal patient tissue, fluids comprising normal cells or cell-free DNA, or other anatomical material. In some implementations, the sample may include abnormal (e.g., cancerous or genetically mutated) tissue, fluids comprising abnormal cells or circulating-tumor DNA, or other anatomical material. In some implementations, the sample may include a combination of normal and abnormal tissue, fluid, or other anatomical material.

The genomic region 100 shown in FIG. 1 may correspond to a single strand or strand fragment of DNA, or a strand or strand fragment of RNA. Although not shown in FIG. 1, the region 100 includes a sequence comprised of various bases (i.e., cytosine (“C”), guanine (“G”), adenine (“A”), thymine (“T”), or uracil (“U”)). The specific sequence of bases can often determine important characteristics of the anatomical material or the patient, e.g. whether the patient has cancer, and, if so, what therapies may be effective or ineffective to treat it.

The techniques described below involve characterizing a sequence of interest 102 within the genomic region 100 as either germline or somatic. The characterization is assisted by use of a reference sequence 104. The reference sequence 104 is an exemplary genomic sequence that represents a “normal” (e.g., non-cancerous) patient. In some implementations, the reference sequence 104 can include a sequence determined by the Human Genome Project, e.g. hg19.

In the reference sequence 104, there are known regions of polymorphism 106a, 106b. A region of polymorphism 106a, 106b is a region (comprising any number of bases from a single base to several hundred or more bases) in which variation of a particular organism's genomic sequence is expected across a population of organisms, without adverse consequences corresponding to the variations. For example, in humans there are regions of polymorphism that correspond to various hair colors, eye colors, or other individualized characteristics. The genomic region 100 corresponding to an actual patient sample will have specific base values 108a, 108b at the positions in the region 100 corresponding to the polymorphic regions 106a, 106b in the reference sequence 104. In other words, the polymorphic regions 106a, 106b of the reference sequence 104 are the locations at which certain characteristics of a person (e.g. hair color) are determined; the base values 108a, 108b are the individualized determinations of those characteristics (e.g., red hair) that describe the specific patient.

In some cases, polymorphic regions 106a, 106b include one or more single nucleotide polymorphisms (or “SNPs”). In some cases, regions of polymorphism can include entire alleles or portions thereof.

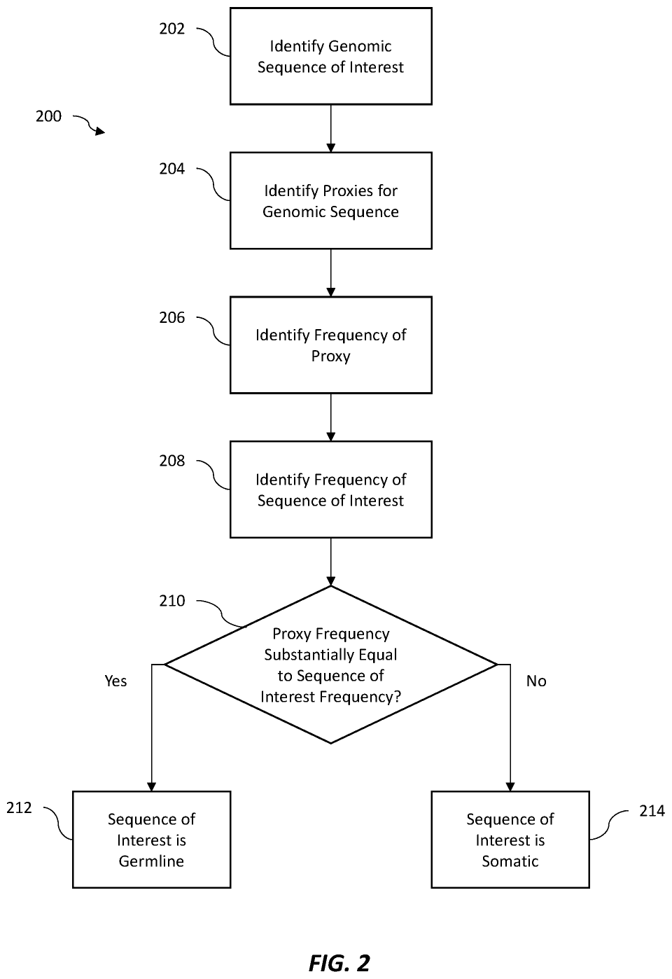

FIG. 2 is a flowchart for a process to distinguish germline and somatic genomic sequences. The process 200 begins with identifying (i.e., selecting or classifying) a genomic region of interest (step 202). In some implementations, step 202 involves identifying a region of interest (i.e., sequence of interest) 102 from within a larger genomic region 100.

Determining a genomic sequence (e.g., a genomic region 100) from a physical sample can be accomplished in a variety of ways. One such way is described in U.S. Pat. No. 9,340,830, and another is described in U.S. Pat. Pub. 2017/0356053, the entireties of both of which are incorporated by reference herein. More generally, there is a category of machines that are operable to determine the genetic sequence of an input sample called genomic sequencers. In some instances, the disclosed methods and systems may be implemented using any of a variety of next generation sequencing (NGS) techniques and sequencers, including cyclic array sequencers configured for massively parallel sequencing and single molecule sequencers. Moreover, there are a variety of known sub-regions of human and other organisms' genomes that are known to be relevant to a variety of medical conditions.

The techniques described herein do not depend on the use of a particular sequencing platform or particular sequencing techniques, and any of these machines and accompanying techniques may be used in step 202. In some instances, the disclosed methods may be implemented using alternative nucleic acid sequence analysis techniques, e.g., microarrays, fluorescence in situ hybridization (FISH), and the like.

In some implementations, the region (i.e., sequence) of interest 102 is identified to correspond to a known genetic locus within a reference genome 104. In some implementations, the region of interest 102 corresponds to a mutation with respect to the reference sequence 104 (i.e., a subsection of the genomic region 100 other than a polymorphic region that has a different genetic sequence from that of the corresponding part of reference sequence 104). In some implementations, the sequence of interest corresponds to a gene relevant to a medical condition that the patient possesses. In some implementations, the region of interest 102 is an oncogene or portion thereof.

In step 204, one or more proxy genomic sequences for the genomic sequence are identified (step 204). The selected one or more proxy genomic sequences may be known germline sequences (for example, based on being matched with a known germline sequence from a database of known germline sequences, or by sequencing healthy tissue, cells, or cell-free DNA from the subject or another healthy individual). Referring to FIG. 1, one characterization of a proxy 110 is a sequence at a genetic locus that is (a) known to encode germline genetic information, and (b) known to have the same copy number as the sequence of interest 102 (for example, because of being physically close to, or confirmed to be located within the same copy number segment as, the sequence of interest 102). An alternative characterization is to require that the proxy 110 is known to encode somatic genetic information. For convenience, this document will assume that proxies 110 encode germline information unless otherwise specified, but those skilled in the art will appreciate the equivalence of the two approaches.

The germline status of a particular candidate proxy sequence may be known from research literature, publicly available databases (e.g., dbSNP (available at www.ncbi.nlm.nih.gov/snp/) or gnomAD (available at gnomad.broadinstitute.org)), or may be discovered by other ab initio means. On the other hand, somatic variants can be identified from matched tumor/normal samples; i.e., samples from the same patient that contain both tumor DNA and non-tumor (“normal”) DNA. In particular, variants seen in tumor DNA but not in corresponding normal DNA are necessarily somatic. Known somatic variants may also be discovered by other ab initio means.

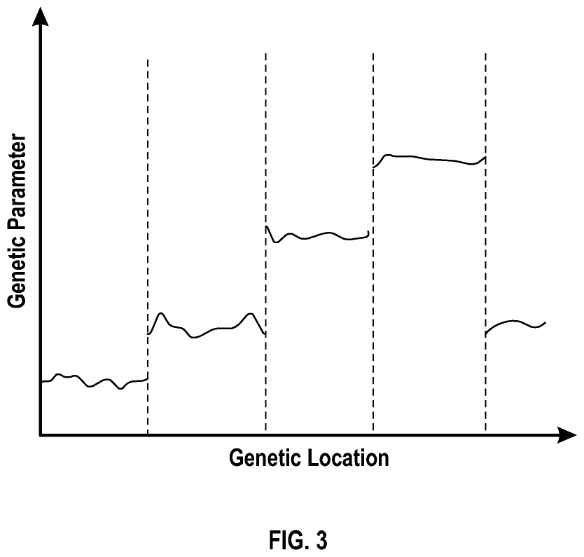

Referring to FIG. 3, in some implementations step 204 is performed by employing a segmentation process. In such a process, the portion 100 of the patient's genome is partitioned into segments (delineated by dashed vertical lines in FIG. 3) based on a genetic parameter. The segments are defined such that the parameter values in a particular segment are all equal (within a desired range, or within a desired threshold). For example, the segment may be a continuous sequence having approximately the same (i.e., within a desired range, or within a desired threshold) sequencing depth or copy number. In some implementations, the genetic parameter used to segment the input includes copy number, frequency of an allele or sub-allelic segment of interest, or others. The one or more proxy sequences may be located within the same segment as the genomic sequence of interest, thus making it highly likely that the one or more proxy genomic sequences and the genomic sequence of interest have the same copy number.

A variety of segmentation procedures are known in the art. For example, iSeg (described in Girimurugan, et al., iSeg: an Efficient Algorithm for Segmentation of Genomic and Epigenomic Data, BMC Bioinformatics 19:131 (2018), the entirety of which is incorporated herein), CBS (described in Olshen, et al., Circular Binary Segmentation for the Analysis of Array-Based DNA Copy Number Data, Biostatistics 2004 October; 5(4):557-72, the entirety of which is incorporated by reference herein), SLMSuite (described in Orlandini, et al., SLMSuite: A Suite of Algorithms for Segmenting Genomic Profiles, BMC Bioinformatics 18:321 (2017), Pelt (described in Killick, et al. Optimal detection of changepoints with a linear computational cost, Journal of the American Statistical Association, 107:500 (2012), the entirety of which is incorporated by reference herein) are four among many such algorithms. In some embodiments, the patient genomic sequence is segmented into a plurality of segments based on copy number uniformity within each segment.

Referring back to FIG. 2, in some implementations only proxies 110 that lie on the same segment as the region of interest 102 are identified. In some implementations the proxies 110 include all known germline SNPs lying on the same segment as the region of interest 102. In some implementations the proxies 110 include all known germline alleles on the same segment as the region of interest 102. In some implementations, e.g., if there is difficulty in correctly segmenting the genome sequence into segments corresponding to different copy numbers, only proxies 110 that are no more than a pre-determined number of bases away from the region of interest 102 are identified. For example, in some instances, the maximum number of bases separating the region of interest from the proxy sequences may range from about 10 bases to about 1,000 bases. In some instances, the maximum number of bases separating the region of interest from the proxy sequences may be about 10 bases, 20 bases, 30 bases, 40 bases, 50 bases, 60 bases, 70 bases, 80 bases, 90 bases, 100 bases, 200 bases, 300 bases, 400 bases, 500 bases, 600 bases, 700 bases, 800 bases, 900 bases, or 1,000 bases. In some instances, the maximum number of bases separating the region of interest from the proxy sequences may have any value within the range of values described in this paragraph.

In step 206, the frequencies of the proxies 110 are identified. In step 208, the allele frequencies (allele fractions) of sequences from the region of interest (i.e., genomic sequence of interest) 102 are identified. Here, “frequency” refers to a normalized statistical frequency—for example, the number of occurrences of a sequence or proxy within the sample, divided by the total number of occurrences of any sequence at the same genomic locus. In some implementations, several frequency measurements may be made. Allele frequencies of the genomic sequence of interest and the one or more proxy genomic sequence can be determined by sequencing the nucleic acid molecules in the sample from the subject. In some instances, allele frequencies may be determined using other methodologies, e.g., microarrays or fluorescence in situ hybridization (FISH) techniques. When using several proxies, outlier proxy frequencies may be discarded and the remaining frequencies may be combined as a single statistical centrality measure (e.g., a summary statistic, such as mean, median, mode, or others, or a distribution (such as a probability distribution) of the allele frequencies of the proxy sequences) so that step 210 involves a single numerical comparison. For example, in some embodiments, the centrality measure (summary statistic) is a mean allele frequency for the one or more proxy sequences. In some embodiments, the centrality measure (summary statistic) is a median allele frequency for the one or more proxy sequences. When a single proxy genomic sequence is used, the centrality measure of observed frequencies of the proxy genomic sequence is the frequency of that proxy sequence. The centrality measure may be, in some embodiments, a distribution of the observed allele frequencies for the proxy sequences.

In decision 210, the proxy frequency or frequencies (for example, a centrality measure of the observed frequencies of the one or more proxy sequences) are compared to the frequency or frequencies of the region of interest to determine if they are equal. Here and throughout this application the term “equal” includes “equal to within a desired range” or “equal to within a desired threshold” that can routinely be determined based on desired selectivity and specificity of the process 200. The range or threshold may be set, for example, using a statistical threshold or statistical test selected by one skilled in the art. If several proxies 110 are used and individual comparisons are made instead of combining the proxy frequencies as described above, then a decision 210 results in a “yes” if a certain proportion of the comparisons (e.g., greater than 50%, greater than 55%, greater than 60%, greater than 65%, greater than 70%, greater than 75%, greater than 80%, greater than 85%, greater than 90%, or greater than 95%) are equal.

If the proxy frequency is equal to the frequency of the sequence of interest, then the sequence of interest is classified as germline (step 212). Otherwise, the sequence of interest is classified as somatic (step 214). Alternatively, if proxies 110 were selected to be known to encode somatic information (instead of germline), then equal frequencies is interpreted as the sequence of interest being somatic and unequal frequencies is interpreted as the sequence of interest being germline.

In some implementations, the comparison in decision 210 may also be used to eliminate potentially erroneous classifications. In particular, the frequency of a true somatic variant is necessarily less than a true germline variant, because both tumor and non-tumor DNA contribute to a germline variant's frequency count, while only tumor DNA contributes to a somatic variant's frequency count. Thus, in some implementations, if the frequency of the sequence of interest is greater than the proxy frequency, then sequence of interest is classified as germline.

By way of example, in some embodiments, comparing the observed frequency of the genomic sequence of interest to the centrality measure of observed frequencies of the one or more proxy genomic sequences can include determining an “allele frequency distance” (AFDIS) of the genomic sequence of interest from the expected allele frequency. The expected allele frequency if the genomic sequence of interest is a germline sequence is determined based on the frequency of the one or more proxy sequences (or summary statistic indicative of the observed frequencies of the one or more proxy sequences), which are assumed to be germline based on the selection of the one or more proxy sequences. The AFDIS may be numerically expressed, in some embodiments, according to

AFDIS=AF_{germline}−AFᵥₐᵣᵢₐₙₜ

wherein AF_{germline} is the expected allele frequency if the genomic sequence of interest were germline, as determined based on the observed allele frequency of the one or more proxy sequences, and AFᵥₐᵣᵢₐₙₜ is the observed allele frequency of the genomic sequence of interest.

In some embodiments, the allele frequency distance may be determined using a distribution of observed frequencies of the proxy genomic sequences. The distribution can be used to determine a probability that the genomic sequence of interest is germline or somatic. In some embodiments, the allele frequency distance is a probability that the observed frequency of the genomic sequence of interest fits within (or does not fit within) the distribution of observed frequencies of a plurality of proxy sequences. For example, if the allele frequency of the genomic sequence of interest fits within the distribution, the genomic sequence of interest may be identified as a germline sequence. If the allele frequency of the genomic sequence of interest does not fit within the distribution, the genomic sequence of interest may be identified as somatic. One skilled in the art may select a statistical test or predetermined threshold to determine if the allele frequency of the genomic sequence of interest fits within the distribution.

In some embodiments, the allele frequency distance may be used to classify the genomic sequence of interest. For example, in some embodiments, if the allele frequency distance is above a selected threshold, the genomic sequence of interest is classified as somatic. In some embodiments, if the allele frequency distance is below a selected threshold, the genomic sequence of interest is classified as germline. The threshold may be set based on the accuracy or specificity tolerance desired.

In some embodiments, classification of the genomic sequence of interest as germline or somatic may include the use of a statistical model. The statistical model can receive, for example, an allele frequency distance for a given genomic sequence of interest, and output a classification of the genomic sequence of interest as somatic (or likely somatic) or germline (or likely germline). The classification may be based on a probability of the genomic sequence of interest being somatic or germline. In some implementations, the genomic sequence of interest may be classified as ambiguous, for example, if the probability of the sequence being somatic or germline is not sufficiently high. The probability threshold for making a call can be based on a desired specificity and/or accuracy of the call. For example, in some embodiments, if the probability of the genomic sequence of interest being somatic is above any one of 0.8, 0.85, 0.9, 0.95, 0.96, 0.97, 0.98, or 0.99 (or any selected value therebetween), the genomic sequence of interest is classified as somatic, and if the probability of the genomic sequence of interest being somatic is below any one of 0.2, 0.15, 0.1, 0.05, 0.04, 0.03, 0.02, or 0.01 (or any selected value therebetween), the genomic sequence of interest is classified as germline. Genomic sequences of interest that are not classified as somatic or germline, based on the statistical model, may be labeled as ambiguous.

In some embodiments, the statistical model is trained using data from one or more matched tumor/normal sample pairs. Normal samples in the matched tumor/normal sample pair can be sequenced to establish a ground truth for germline sequences, and the tumor sample can be sequenced to establish a ground truth for somatic variant sequences (i.e., those sequences that are not germline according to the matched normal sample). Sequencing data from the tumor sample, which can include a mixture of normal and tumor nucleic acid molecules, can be used to determine allele frequency distances for selected genomic sequences of interest, which are then labeled as somatic (probability of being somatic, p_{somatic}, being equal to 1) or germline (p_{somatic} being equal to 0). A function associating allele frequency distance to probability of being somatic can then be generated using the training data.

Other methods of training the statistical model may be used. For example, in some embodiments, the model is trained using only data for germline sequences or only data for somatic sequences.

In some implementations, the comparison of step 210 may be indirectly performed by way of a statistical model. For example, if the median allele frequency of a collection of proxies is used as the central measure of step 206, then a logistic regression model may be constructed that describes the difference of the allele frequency of the sequence of interest from the median allele frequency of the proxies. In some implementations, this logistic regression model can be constructed from data for a collection of matched tumor/normal samples, such that the difference described in the previous sentence is proportional to log

$$( \frac{p}{1 - p} ) ,$$

where p represents the probability that the sequence of interest comprises a somatic variant.

The rationale underlying this characterization is that each proxy is physically close to the sequence of interest in the patient's genome. Thus, it is likely that the proxy and the sequence of interest experience the same or similar genomic dynamics or mutations, such as duplication events or deletions. Rather than attempting to model the specific dynamics of the sequence of interest to correlate observed frequencies with germline/somatic status, this approach replaces such a model with a direct empirical measurement. Insofar as prior art models have historically been insensitive or inaccurate to some degree, this approach provides an advantage.

The methods described herein may further include generating a report that indicates one or more genomic sequences of interest as germline or somatic. The generated report can be transmitted to the patient, healthcare providers, or others (for example, using a computer network). The report is particularly beneficial for evaluating cancer treatment therapies, making treatment decisions, monitoring cancer progression or recurrence, designing personalized cancer vaccine, and other beneficial uses.

### Electronic Devices and Systems

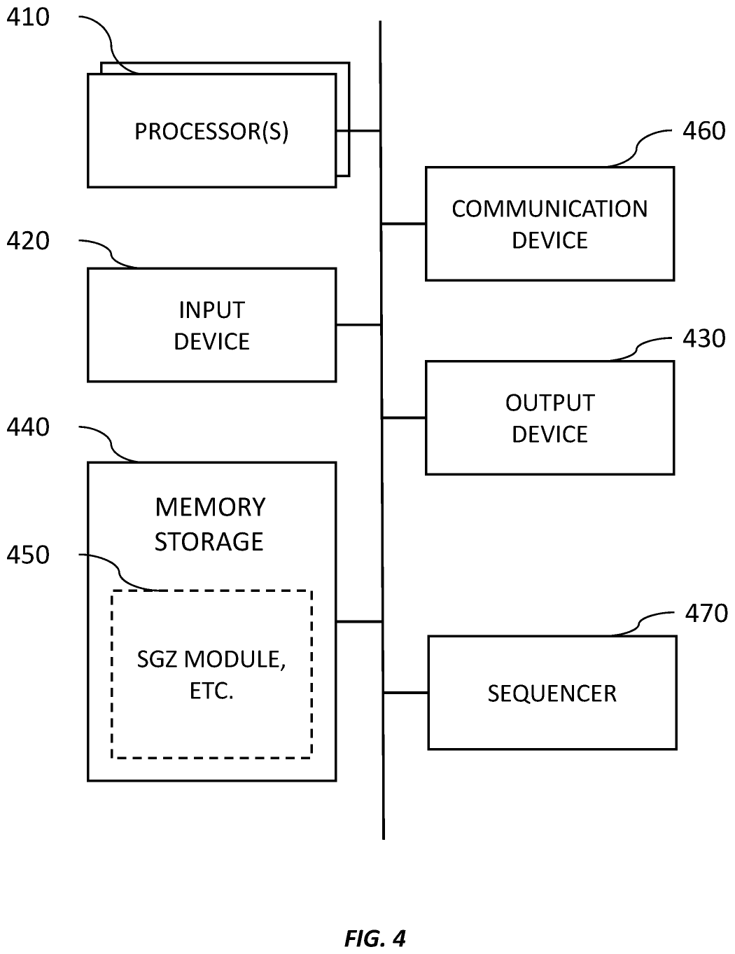

FIG. 4 illustrates an example of a system in accordance with one embodiment. Device 400 can be a host computer connected to a network. Device 400 can be a client computer or a server. As shown in FIG. 4, device 400 can be any suitable type of microprocessor-based device, such as a personal computer, workstation, server or handheld computing device (portable electronic device) such as a phone or tablet. The device can include, for example, one or more processors 410, an input device 420, an output device 430, a memory storage 440, and/or a communication device 460. Input device 420 and output device 430 can either be connectable or integrated with the computer. In some embodiments, the device is configured to operate a sequencer 470, which can sequence nucleic acid molecules in a patient sample to obtain sequencing data.

Input device 420 can be any suitable device that provides input, such as a touch screen, keyboard or keypad, mouse, or voice-recognition device. Output device 430 can be any suitable device that provides output, such as a display, touch screen, haptics device, or speaker.

Memory storage 440 can be any suitable device that provides storage, such as an electrical, magnetic or optical memory including a RAM, cache, hard drive, or removable storage disk. Communication device 460 can include any suitable device capable of transmitting and receiving signals over a network, such as a network interface chip or device. The components of the computer can be connected in any suitable manner, such as via a physical bus or wirelessly.

Software, such as the SGZ module 450 and other sequence analysis and variant calling program modules, which can be stored in memory storage 440 and executed by processor(s) 410, can include, for example, code for the AFDIS-based logistic regression models and other programming that embodies the functionality of the present disclosure (e.g., as embodied in the devices as described above).

Software, such as the SGZ module 450 and other sequence analysis and variant calling program modules, can also be stored and/or transported within any non-transitory computer-readable storage medium for use by or in connection with an instruction execution system, apparatus, or device, such as those described above, that can fetch instructions associated with the software from the instruction execution system, apparatus, or device and execute the instructions. In the context of this disclosure, a computer-readable storage medium can be any medium, such as storage 440, that can contain or store programming for use by or in connection with an instruction execution system, apparatus, or device.

Software, such as the SGZ module 450 and other sequence analysis and variant calling program modules, can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport readable medium can include, but is not limited to, an electronic, magnetic, optical, electromagnetic or infrared wired or wireless propagation medium.

Device 400 may be connected to a network, which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 400 can implement any operating system suitable for operating on the network. Software, such as the SGZ module 450 and other sequence analysis and variant calling program modules, can be written in any suitable programming language, such as C, C++, Java or Python. In various embodiments, application software embodying the functionality of the present disclosure can be deployed in different configurations, such as in a client/server arrangement or through a Web browser as a Web-based application or Web service, for example.

### Subjects, Samples, and Sequencing

The subject samples (e.g., patient samples) used with the methods described herein may include a mixture of tumor and non-tumor nucleic acid molecules. The tumor nucleic acid molecules may be obtained directly or indirectly from the tumor. For example, the tumor nucleic acid molecules may be obtained from a tissue biopsy of a tumor. Tumor biopsies often include both tumor and non-tumor tissue, thereby providing a mixture of tumor and non-tumor nucleic acid molecules. In some embodiments, the tumor and non-tumor nucleic acid molecules are obtained from a bodily fluid or liquid biopsy sample (e.g., blood, plasma, spinal fluid, etc.), that may include cell-free (or circulating free) DNA including tumor (e.g., circulating tumor DNA, or ctDNA) and non-tumor cell-free nucleic acid molecules.

The patient sample may be taken, for example, from a subject with cancer, a subject suspected of having cancer, or a subject having previously been treated for a cancer. In certain embodiments, the sample is acquired from a subject having a solid tumor, a hematological cancer, or a metastatic form thereof. In certain embodiments, the sample is obtained from a subject having a cancer, or at risk of having a cancer. In certain embodiments, the sample is obtained from a subject who has not received a therapy to treat a cancer, is receiving a therapy to treat a cancer, or has received a therapy to treat a cancer, as described herein.

A variety of tissues can be the source of the samples used in the present methods. Genomic or subgenomic nucleic acid (e.g., DNA or RNA) can be isolated from a subject's sample (e.g., a sample comprising tumor cells, a blood sample, a blood constituent sample, a sample comprising cell-free DNA (cfDNA), a sample comprising circulating tumor DNA (ctDNA), a sample comprising circulating tumor cells (CTCs), or any normal control (e.g., a normal adjacent tissue (NAT)).

In some embodiments, the sample is acquired from a liquid biopsy. A liquid biopsy patient sample may be derived from, for example, blood, plasma, cerebrospinal fluid, sputum, stool, urine, or saliva.

In some embodiments, the patient sample is derived from a solid tissue sample, such as a solid tumor biopsy. Solid tumor biopsies often include a mixture of tumor and non-tumor tissue. In some embodiments, the solid tissue biopsy sample is a fresh sample. In some embodiments, the solid tissue biopsy sample is a frozen sample or previously frozen sample. In some embodiments, the solid tissue biopsy sample is a fresh sample. In some embodiments, the solid tissue biopsy sample is a preserved sample (for example, a chemically preserved sample). In certain embodiments, the sample is a formalin-fixed paraffin-embedded (FFPE) sample.

In some embodiments, the tumor purity of the patient sample (i.e., the portion of the sample that is tumor nucleic acid molecules compared to total nucleic acid molecules) for any of the sample types disclosed herein is about 1% or more, about 5% or more, about 10% or more, about 15% or more, about 20% or more, about 25% or more, about 30% or more, about 40% or more, about 50% or more, about 60% or more, about 70% or more, or about 80% or more. In some embodiments, the tumor purity of the patient sample is about 99% or less, about 95% or less, about 90% or less, about 85% or less, about 80% or less, about 75% or less, about 70% or less, about 60% or less, about 50% or less, about 40% or less, about 30% or less, about 25% or less, or about 20% or less.

In one embodiment, the method further includes obtaining a sample, e.g., a patient sample described herein. The sample can be acquired directly or indirectly. In an embodiment, the sample is acquired, e.g., by isolation or purification, from a sample that comprises cfDNA. In an embodiment, the sample is acquired, e.g., by isolation or purification, from a sample that comprises ctDNA. In an embodiment, the sample is acquired, e.g., by isolation or purification, from a sample that comprises both malignant cells and non-malignant cells (e.g., tumor-infiltrating lymphocyte). In an embodiment, the sample is acquired, e.g., by isolation or purification, from a sample that comprises CTCs. In some embodiments, the sample is obtained by a solid tissue biopsy.

A sequencing library can be prepared from a patient sample using known methods. The nucleic acid molecules may be purified or isolated from the patient sample. In some embodiments, the isolated nucleic acids are fragmented or sheared using a known method. For example, nucleic acid molecules may be fragmented by physical shearing methods (e.g., sonication), enzymatic cleavage methods, chemical cleavage methods, and other methods well known to those skilled in the art. The nucleic acid may be ligated to an adapter sequence for sequencing. In some instances, the adapter may comprise an amplification primer and/or sequencing adapter. In some instances, nucleic acid molecules purified or isolated from the patient sample, or the sequencing library prepared therefrom, may be amplified, e.g., using a polymerase chain reaction (PCR) or isothermal amplification method known to those of skill in the art.

In some embodiments, the nucleic acid molecules from the patient sample and used to prepare a sequencing library (or a selected (e.g., captured) subset thereof) are sequenced to generate a patient genomic sequence. Sequencing methods are well known in the art, and may be performed using multiplexed (e.g., next-generation) or single molecule sequencing. The patient genomic sequence determined by sequencing need not be the full genome of the patient. For example, in some embodiments, targeted sequencing methods (e.g., using specific probes (or bait) molecules for hybridization-based capture) are used to sequence portions of the patient's genome (i.e., less than the full genome). See, for example, U.S. Pat. No. 9,340,830 B2. Targeted sequencing may be used to target, for example, one or more exon regions, one or more intron regions, one or more intragenic regions, one or more 3′-UTRs (untranslated regions), and/or one or more 5′-UTRs.

In some embodiments, targeted sequencing may be used to sequence one or more genes, or portions of one or more genes, associated with cancer. Exemplary genes associated with cancer that may be sequenced using targeted sequencing include, but are not limited to ABL2, AKT2, AKT3, ARAF, ARFRP1, ARID1A, ATM, ATR, AURKA, AURKB, BCL2, BCL2A1, BCL2L1, BCL2L2, BCL6, BRCA1, BRCA2, CARD11, CBL, CCND1, CCND2, CCND3, CCNE1, CDH1, CDH2, CDH20, CDH5, CDK4, CDK6, CDK8, CDKN2B, CDKN2C, CHEK1, CHEK2, CRKL, CRLF2, DNMT3A, DOT1L, EPHA3, EPHA5, EPHA6, EPHA7, EPHB1, EPHB4, EPHB6, ERBB3, ERBB4, ERG, ETV1, ETV4, ETV5, ETV6, EWSR1, EZH2, FANCA, FBXW7, FGFR4, FLT1, FLT4, FOXP4, GATA1, GNA11, GNAQ, GNAS, GPR124, GUCY1A2, HOXA3, HSP90AA1, IDH1, IDH2, IGF1R, IGF2R, IKBKE, IKZF1, INHBA, IRS2, JAK1, JAK3, JUN, KDR, LRP1B, LTK, MAP2K1, MAP2K2, MAP2K4, MCL1, MDM2, MDM4, MEN1, MITF, MLH1, MPL, MRE11A, MSH2, MSH6, MTOR, MUTYH, MYCL1, MYCN, NF2, NKX2-1, NTRK1, NTRK3, PAK3, PAX5, PDGFRB, PIK3R1, PKHD1, PLCG1, PRKDC, PTCH1, PTPN11, PTPRD, RAF1, RARA, RICTOR, RPTOR, RUNX1, SMAD2, SMAD3, SMAD4, SMARCA4, SMARCB1, SMO, SOX10, SOX2, SRC, STK11, TBX22, TET2, TGFBR2, TMPRSS2, TOP1, TSC1, TSC2, USP9X, VHL, WT1, ABL1, AKT1, ALK, APC, AR, BRAF, CDKN2A, CEBPA, CTNNB1, EGFR, ERBB2, ESR1, FGFR1, FGFR2, FGFR3, FLT3, HRAS, JAK2, KIT, KRAS, MET, MLL, MYC, NF1, NOTCH1, NPM1, NRAS, PDGFRA, PIK3CA, PTEN, RB1, RET, and TP53.

In certain embodiments, the sample is acquired from a subject having a cancer. Exemplary cancers include, but are not limited to, B cell cancer, e.g., multiple myeloma, melanomas, breast cancer, lung cancer (such as non-small cell lung carcinoma or NSCLC), bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematological tissues, adenocarcinomas, inflammatory myofibroblastic tumors, gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancers, essential thrombocythemia, agnogenic myeloid metaplasia, hypereosinophilic syndrome, systemic mastocytosis, familiar hypereosinophilia, chronic eosinophilic leukemia, neuroendocrine cancers, carcinoid tumors, and the like.

In an embodiment, the cancer is a hematologic malignancy (or premaligancy). As used herein, a hematologic malignancy refers to a tumor of the hematopoietic or lymphoid tissues, e.g., a tumor that affects blood, bone marrow, or lymph nodes. Exemplary hematologic malignancies include, but are not limited to, leukemia (e.g., acute lymphoblastic leukemia (ALL), acute myeloid leukemia (AML), chronic lymphocytic leukemia (CLL), chronic myelogenous leukemia (CML), hairy cell leukemia, acute monocytic leukemia (AMoL), chronic myelomonocytic leukemia (CMML), juvenile myelomonocytic leukemia (JMML), or large granular lymphocytic leukemia), lymphoma (e.g., AIDS-related lymphoma, cutaneous T-cell lymphoma, Hodgkin lymphoma (e.g., classical Hodgkin lymphoma or nodular lymphocyte-predominant Hodgkin lymphoma), mycosis fungoides, non-Hodgkin lymphoma (e.g., B-cell non-Hodgkin lymphoma (e.g., Burkitt lymphoma, small lymphocytic lymphoma (CLL/SLL), diffuse large B-cell lymphoma, follicular lymphoma, immunoblastic large cell lymphoma, precursor B-lymphoblastic lymphoma, or mantle cell lymphoma) or T-cell non-Hodgkin lymphoma (mycosis fungoides, anaplastic large cell lymphoma, or precursor T-lymphoblastic lymphoma)), primary central nervous system lymphoma, Sézary syndrome, Waldenström macroglobulinemia), chronic myeloproliferative neoplasm, Langerhans cell histiocytosis, multiple myeloma/plasma cell neoplasm, myelodysplastic syndrome, or myelodysplastic/myeloproliferative neoplasm. Premaligancy, as used herein, refers to a tissue that is not yet malignant but is poised to become malignant.

In some embodiments, the sample is obtained, e.g., collected, from a subject, e.g., patient, with a condition or disease, e.g., a hyperproliferative disease (e.g., as described herein) or a non-cancer indication. In some embodiments, the disease is a hyperproliferative disease. In some embodiments, the hyperproliferative disease is a cancer, e.g., a solid tumor or a hematological cancer. In some embodiments, the cancer is a solid tumor. In some embodiments, the cancer is a hematological cancer, e.g. a leukemia or lymphoma.

In some embodiments, the subject has a cancer. In some embodiments, the subject has been, or is being treated, for cancer. In some embodiments, the subject is in need of being monitored for cancer progression or regression, e.g., after being treated with a cancer therapy. In some embodiments, the subject is in need of being monitored for relapse of cancer. In some embodiments, the subject is at risk of having a cancer. In some embodiments, the subject has not been treated with a cancer therapy. In some embodiments, the subject has a genetic predisposition to a cancer (e.g., having a mutation that increases his or her baseline risk for developing a cancer). In some embodiments, the subject has been exposed to an environment (e.g., radiation or chemical) that increases his or her risk for developing a cancer. In some embodiments, the subject is in need of being monitored for development of a cancer.

In some embodiments, the patient has been previously treated with a targeted therapy, e.g., one or more targeted therapies. In some embodiments, for a patient who has been previously treated with a targeted therapy, a post-targeted therapy sample, e.g., specimen is obtained, e.g., collected. In some embodiments, the post-targeted therapy sample is a sample obtained, e.g., collected, after the completion of the targeted therapy.

In some embodiments, the patient has not been previously treated with a targeted therapy. In some embodiments, for a patient who has not been previously treated with a targeted therapy, the sample comprises a resection, e.g., an original resection, or a recurrence, e.g., disease recurrence post-therapy, e.g., non-targeted therapy. In some embodiments, the sample is or is part of a primary tumor or a metastasis, e.g., metastasis biopsy. In some embodiments, the sample is obtained from a site, e.g., tumor site, with the highest percent of tumor, e.g., tumor cells, as compared to adjacent sites, e.g., adjacent sites with tumor cells. In some embodiments, the sample is obtained from a site, e.g., tumor site, with the largest tumor focus as compared to adjacent sites, e.g., adjacent sites with tumor cells.

In some embodiments, the subject is a human.

### Methods of Treating Cancer

The genomic profile of a cancer can often affects the likelihood of success of various cancer treatment modalities. For example, a given anti-cancer agent may be more likely to successfully treat a particular cancer having one genomic profile versus another. The methods described herein can be used characterize the genomic profile of a cancer by distinguishing somatic sequences, which may be attributed to the cancer, from germline sequences.

By way of example, a method of treating cancer in a patient can include identifying (e.g., classifying) one or more genomic sequences of interest as somatic using a method described herein, and selecting a cancer treatment modality based on the one or more identified somatic sequences. The cancer can then be treated using an effective amount of the selected cancer treatment modality. This allows for personalized cancer treatment of the patient based on the somatic sequences specific to that patient's cancer. In contrast, if the treatment selection was based on a germline variant rather than a somatic variant, there is some risk that the selected treatment modality may be ineffective for the patient's cancer.

Exemplary cancer treatment modalities may include, for example, a selected chemotherapeutic agent, a selected immune-oncology agent (such as an immune checkpoint inhibitor), resection surgery, radiation therapy, targeted therapy, gene expression modulators, angiogenesis inhibitors, and hormone therapy, among others.

The cancer treatment may be selected, for example, based on an association between the one or more identified somatic sequences and successful cancer treatment using the selected treatment modality. Exemplary associations between cancer type, somatic sequence, and treatment modality are listed in Table 1.

TABLE 1

| Indication | Somatic Sequence(s) | Treatment Modality |
|---|---|---|
| Non-small cell lung cancer | EGFR exon 19 deletion | Administration of an |
| (NSCLC) | and/or EGFR exon 21 | effective amount of a |
| | L858R alteration | protein kinase inhibitor |
| | | (e.g., afatinib, erlotinib, or |
| | | gefitinib) |
| Non-small cell lung cancer | EGFR exon 18 G719 | Administration of an |
| (NSCLC) | alteration | effective amount of a |
| | | protein kinase inhibitor |
| | | (e.g., afatinib, erlotinib, or |
| | | gefitinib) |
| Non-small cell lung cancer | EGFR exon 20 T790M | Administration of an |
| (NSCLC) | alteration | effective amount of a |
| | | EGFR tyrosine kinase |
| | | inhibitor (e.g., osimertinib) |
| Non-small cell lung cancer | ALK rearrangement | Administration of an |
| (NSCLC) | | effective amount of an |
| | | ALK inhibitor (e.g., |
| | | alectinib, crizotinib, or |
| | | ceritinib) |
| Non-small cell lung cancer | BRAF V600E | Administration of an |
| (NSCLC) | | effective amount of a B- |
| | | Raf inhibitor (e.g., |
| | | dabrafenib) and/or a MEK |
| | | inhibitor (e.g., trametinib) |
| Non-small cell lung cancer | MET single nucleotide | Administration of an |
| (NSCLC) | variants (SNVs) and/or an | effective amount of a |
| | indel that leads to MET | c-Met inhibitor (e.g., |
| | exon 14 skipping | capmatinib) |
| Melanoma | BRAF V600E | Administration of an |
| | | effective amount of a B- |
| | | Raf inhibitor (e.g., |
| | | dabrafenib or vemurafenib) |
| Melanoma | BRAF V600E orV600K | Administration of an |
| | | effective amount of a MEK |
| | | inhibitor (e.g., trametinib |
| | | or cobimetinib) and/or a B- |
| | | Raf inhibitor (e.g., |
| | | vemurafenib) |
| Breast Cancer | PIK3CA C420R, E542K, | Administration of an |
| | E545A, E545D | effective amount of an |
| | (1635G > T), E545G, | alpha-specific PI3K |
| | E545K, Q546E, Q546R, | inhibitor (e.g., alpelisib) |
| | H1047L, H1047R, and/or | |
| | H1047Y alteration | |
| Ovarian cancer | BRCA1 and/or BRCA2 | Administration of an |
| | alteration | effective amount of a |
| | | PARP inhibitor (e.g., |
| | | olaparib or rucaparib) |
| Cholangiocarcinoma | FGFR2 fusion or | Administration of an |
| | rearrangement | effective amount of an |
| | | FGFR tyrosine kinase |
| | | inhibitor (e.g., |
| | | pemigatinib) |
| Prostate cancer | Homologous | Administration of an |
| | Recombination Repair | effective amount of a |
| | (HRR) gene alteration | PARP inhibitor (e.g., |
| | (e.g., an alteration in one or | olaparib) |
| | more of BRCA1, BRCA2, | |
| | ATM, BARD1, BRIP1, | |
| | CDK12, CHEK1, CHEK2, | |
| | FANCL, PALB2, RAD51B, | |
| | RAD51C, RAD51D, or | |
| | RAD54L) | |

Microsatellite instability (MSI) status of a cancer can be useful for selecting treatment modality of the cancer. Microsatellite instability can result from deficient DNA mismatch repair (MMR) pathways in a cancer cell, which results in an abnormally high frequency of genetic mutations. See Kim, et al., The Landscape of Microsatellite Instability in Colorectal and Endometrial Cancer Genomes, Cell, vol., 155, no. 4, pp. 858-868 (2013). MSI status is generally characterized as being high (MSI-H), low (MSI-L), or stable (MSS) (or, alternatively, MSI-H or not MSI-H; or MSI-H or MSI-undetermined) based on MSI signatures. MSI-H status has been detected for multiple types of solid tumors, and may be an indicator of successful cancer treatment using certain cancer treatment modalities. See Cortes-Ciriano, et al., A molecular portrait of microsatellite instability across multiple cancers, Nature Communications, vol. 8, no. 15180 (2017). Mutations in the microsatellites (i.e., MSI events) can be detected by distinguishing somatic sequences from germline sequences using the methods described herein.

Success of certain cancer treatment modalities has been associated with MSI-H status of a cancer. For example, a PD-1 inhibitor (namely, pembrolizumab) has been found to be particularly effective in treating MSI-H solid tumors (for example, unresectable or metastatic solid tumors). In some embodiments, the cancer determined to have an MSI-H status is treated with an effective amount of an immune-oncology agent. In some embodiments, the cancer determined to have an MSI-H status is treated with an effective amount of an immune checkpoint inhibitor. In some embodiments, the immune checkpoint inhibitor is AMP-224, AMP-514, atezolizumab, AUNP12, avelumab, BGB-A317, BMS-986189, CA-170, camrelizumab, cemiplimab, CK-301, dostarlimab, durvalumab, ipilimumab, INCMGA00012, KN035, nivolumab, pembrolilzumab, sintilimab, spartalizumab, tislelizumab, or toripalimab. In some embodiments, the cancer determined to have an MSI-H status is treated with an effective amount of a PD-1 inhibitor, a PD-L1 inhibitor, or a CTLA-4 inhibitor. In some embodiments, the cancer determined to have an MSI-H status is treated with an effective amount of pembrolizumab.

In some embodiments, the method of treating cancer includes identifying (e.g., classifying) one or more genomic sequences of interest as somatic using the method described herein; determining a microsatellite instability status of the cancer using the identified somatic sequences; and selecting a cancer treatment modality based on the microsatellite instability status of the cancer. The cancer can then be treated using an effective amount of the selected cancer treatment modality. In some embodiments, the cancer is colorectal cancer, endometrial cancer, biliary cancer, bladder cancer, breast cancer, esophageal cancer, gastric cancer, gastroesophageal junction cancer, pancreatic cancer, prostate cancer, renal cell cancer, retroperitoneal adenocarcinoma, sarcoma, small cell lung cancer, small intestinal cancer, or thyroid cancer.

In some embodiments, tumor mutational burden (TMB) of the cancer is determined using one or more somatic sequences identified using the method described herein to select a treatment modality. TMB is a genomic biomarker for the cancer that quantifies the frequency of somatic mutations in a patient's tumor. TMB-high correlates with higher neoantigen expression, which helps the immune system recognize tumors. It has been detected across numerous tumor types and has been associated with improved response rate and prolonged progression-free survival for patients on immunotherapy. See Goodman, et al., Tumor Mutational Burden as an Independent Predictor of Response to Immunotherapy in Diverse Cancers, Mol. Cancer Ther., vol. 16, no. 11, pp. 2598-2608 (2017).

The tumor mutational burden can be determined for a cancer by identifying somatic sequences associated with the cancer using the method described herein.

TMB can provide a quantitative value such that a cancer treatment modality may be selected based on the tumor mutational burden being above or below a predetermined tumor mutational burden threshold. In some embodiments, the predetermined threshold is about 5 mutations/Mb, about 10 mutations/Mb, about 15 mutations/Mb, about 20 mutations/Mb, about 25 mutations/Mb, about 30 mutations/MB, about 40 mutations/Mb, about 50 mutations/Mb, or higher, or any number therebetween (for example, the predetermined threshold may be between 5 mutations/Mb and about 50 mutations/Mb). By way of example, certain immune-oncology agents have been found to be particularly effective when used to treat tumors having a high tumor mutational burden. See, for example, Fabrizio, et al., Beyond microsatellite testing: assessment of tumor mutational burden identifies subsets of colorectal cancer who may respond to immune checkpoint inhibition, J. Gastrointestinal Oncology, vol. 9, no. 4, pp. 610-617 (2018).

In some embodiments, the cancer determined to have a TMB above a predetermined threshold is treated with an effective amount of an immune-oncology agent. In some embodiments, the cancer determined to have a TMB above a predetermined threshold is treated with an effective amount of an immune checkpoint inhibitor. In some embodiments, the immune checkpoint inhibitor is AMP-224, AMP-514, atezolizumab, AUNP12, avelumab, BGB-A317, BMS-986189, CA-170, camrelizumab, cemiplimab, CK-301, dostarlimab, durvalumab, ipilimumab, INCMGA00012, KN035, nivolumab, pembrolilzumab, sintilimab, spartalizumab, tislelizumab, or toripalimab. In some embodiments, the cancer determined to have a TMB above a predetermined threshold is treated with an effective amount of a PD-1 inhibitor, a PD-L1 inhibitor, or a CTLA-4 inhibitor. In some embodiments, the cancer determined to have a TMB above a predetermined threshold is treated with an effective amount of pembrolizumab. In some embodiments, the cancer determined to have a TMB above a predetermined threshold is treated with an effective amount of pembrolizumab, wherein the predetermined threshold is about 10 mutations/Mb.

In some embodiments, the method of treating cancer includes identifying one or more genomic sequences of interest as somatic using the method described herein; determining a tumor mutational burden for the cancer using the one or more identified somatic sequences; and selecting the cancer treatment modality based on the tumor mutational burden being above a predetermined tumor mutational burden threshold. The cancer can then be treated using an effective amount of the selected cancer treatment modality. In some embodiments, the cancer is colorectal cancer, endometrial cancer, biliary cancer, bladder cancer, breast cancer, esophageal cancer, gastric cancer, gastroesophageal junction cancer, pancreatic cancer, prostate cancer, renal cell cancer, retroperitoneal adenocarcinoma, sarcoma, small cell lung cancer, small intestinal cancer, or thyroid cancer.

### Monitoring Cancer Progression

Cancer progression monitoring and/or minimum residual disease detection is beneficial for evaluating a cancer treatment plan and/or monitoring a patient for cancer recurrence. A cancer patient may be treated for a cancer to a point where the cancer is no longer detectable. Nevertheless, the patient may remain susceptible to recurrence. The patient may be monitored for cancer recurrence by detecting nucleic acid molecules derived from a recurring tumor (for example, ctDNA molecules). In other embodiments, a cancer patient may be treated for a disease, and progression of the cancer (e.g., an increase or decrease in the amount of cancer) may be monitored by quantifying the amount of detected tumor nucleic acid molecules in the patient (e.g., a ctDNA level).

Identification of somatic sequences may be particularly useful in monitoring cancer progression or detecting minimum residual disease of a cancer. The somatic sequences provide a genomic signature for the cancer, and they can be used to distinguish tumor nucleic acid molecules from non-tumor nucleic acid molecules.

Patient samples may be obtained and analyzed at two or more time points to monitor cancer progression nor recurrence of the cancer. A first sample is analyzed to identify one or more somatic sequences according to the methods described herein. The first sample may be obtained before, during, or after cancer treatment, although the patient generally has some amount of detectable cancer.

A second sample may be obtained at a later time point after the patient has been treated for the cancer, and can be analyzed to determine if the one or more of the identified somatic sequences are present in the sample. The presence of the somatic sequences indicates that the patient still has the cancer or that the cancer has recurred. Failure to detect the somatic sequences does not definitively prove that the patient is free from cancer, but indicates that the cancer level may be low.

The second patient sample may be the same type of sample as the first patient sample type, or may be a different sample type. In some embodiments, the second patient sample is obtained from a liquid biopsy. For example, the liquid biopsy patient sample may be blood, plasma, cerebrospinal fluid, sputum, stool, urine, or saliva. In some embodiments, the patient sample is obtained from a solid tissue sample such as a solid tumor biopsy. In some embodiments, the solid tissue biopsy sample is a fresh sample. In some embodiments, the solid tissue biopsy sample is a frozen sample or previously frozen sample. In some embodiments, the solid tissue biopsy sample is a fresh sample. In some embodiments, the solid tissue biopsy sample is a preserved sample (for example, a chemically preserved sample). In certain embodiments, the sample is a formalin-fixed paraffin-embedded (FFPE) sample.

The somatic sequences may be detected in DNA or RNA (or both) from the second sample. The presence or absence of the somatic sequences in the second sample may be detected by sequencing, quantitative PCR (qPCR), reverse-transcription PCR (RT-PCR), fluorescent in situ hybridization (FISH), or any other suitable method of specific detection of the one or more somatic sequences. In certain embodiments, the nucleic acid molecules are isolated form the second sample. In some embodiments, the nucleic acid molecules are detected directly from the second sample.

In some embodiments, the presence of the one or more somatic sequences are identified in the second sample, the patient may be treated for cancer using the same treatment modality or a different treatment modality for which the cancer was previously treated.

In some embodiments, a method of monitoring cancer progression or recurrence in a patient includes identifying one or more genomic sequences of interest as somatic using the method a method described herein, wherein the patient sample is obtained from a patient having cancer; obtaining a second patient sample from the patient after the cancer has been treated; and detecting the presence or absence of the one or more genomic sequences of interest identified as somatic within the second patient sample. For example, the one or more genomic sequences of interest may be identified as somatic by selecting a genomic sequence of interest at a genomic locus from within a patient genomic sequence obtained for a patient sample comprising a mixture of tumor nucleic acid molecules and non-tumor nucleic acid molecules; selecting one or more proxy genomic sequences for the genomic sequence of interest; determining an allele frequency distance using an observed allele frequency of the genomic sequence of interest and a summary statistic indicative of observed frequencies of the one or more proxy genomic sequences; and identifying the genomic sequence of interest as germline or somatic using the allele frequency distance. In some embodiments, the method comprises treating the cancer in the patient after the first patient sample is obtained from the patient and before the second patient sample is obtained from the patient. In some embodiments, the method comprises treating the cancer in the patient if the presence of the one or more genomic sequences of interest identified as somatic are detected within the second patient sample.

### Neoantigen Selection and Cancer Vaccine Production

Somatic sequences detected in exon regions of various genes may be suitable as a neoantigen, for example in the development of a personalized cancer vaccine. Peptides can be generated based on the nucleic acid sequence encoded by the somatic variant sequence, which can stimulate the immune system to kill the cancer cells. See, for example, Richters, et al., Best practices for bioinformatics characterization of neoantigens for clinical utility, Genome Medicine, vol., 11 no. 56 (2019).

In some embodiments, a method of selecting a neoantigen for a cancer vaccine personalized for a subject having cancer includes identifying one or more genomic sequences of interest as somatic using the method described herein, wherein the one or more genomic sequences of interest identified as somatic is located within an exon region of a gene; and selecting, from the one or more genomic sequences of interest identified as somatic, a genomic sequence that encodes a neoantigen suitable as a cancer vaccine for the subject. For example, the one or more genomic sequences of interest may be identified as somatic by selecting a genomic sequence of interest at a genomic locus from within a patient genomic sequence obtained for a patient sample comprising a mixture of tumor nucleic acid molecules and non-tumor nucleic acid molecules; selecting one or more proxy genomic sequences for the genomic sequence of interest; determining an allele frequency distance using an observed allele frequency of the genomic sequence of interest and a summary statistic indicative of observed frequencies of the one or more proxy genomic sequences; and identifying the genomic sequence of interest as germline or somatic using the allele frequency distance.

In some embodiments, the method further comprises making a vaccine comprising the neoantigen.

### EXAMPLES

### Example 1—Discrimination Between Somatic and Germline Variants Based on Allele Frequency Distance (AFDIS)

The following example is provided to illustrate an exemplary embodiment of the invention described herein, and is not intended to limit the scope of the invention.

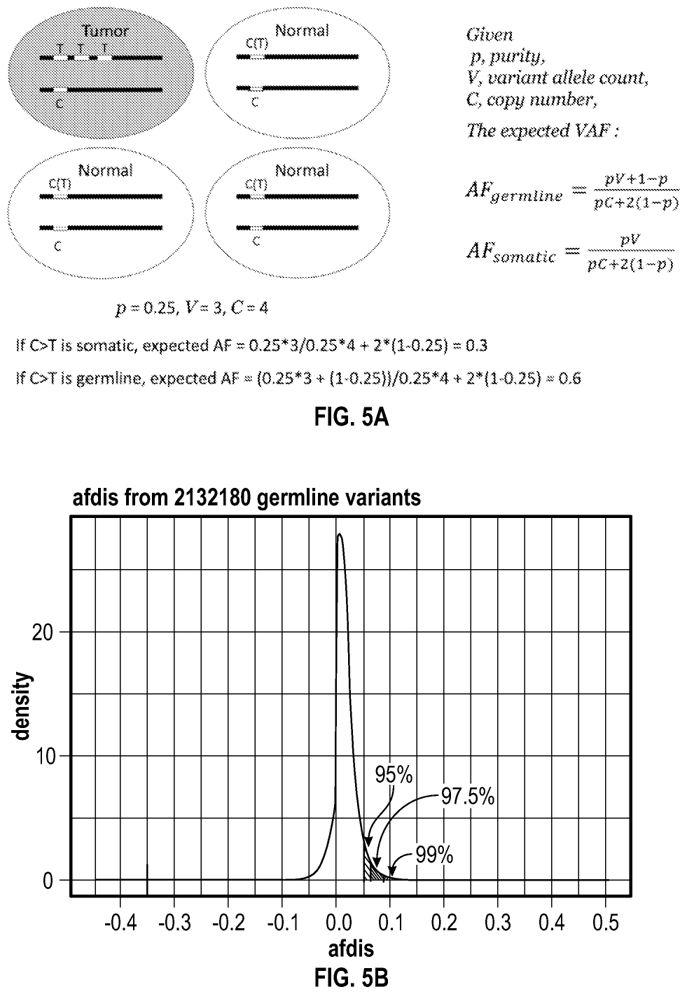

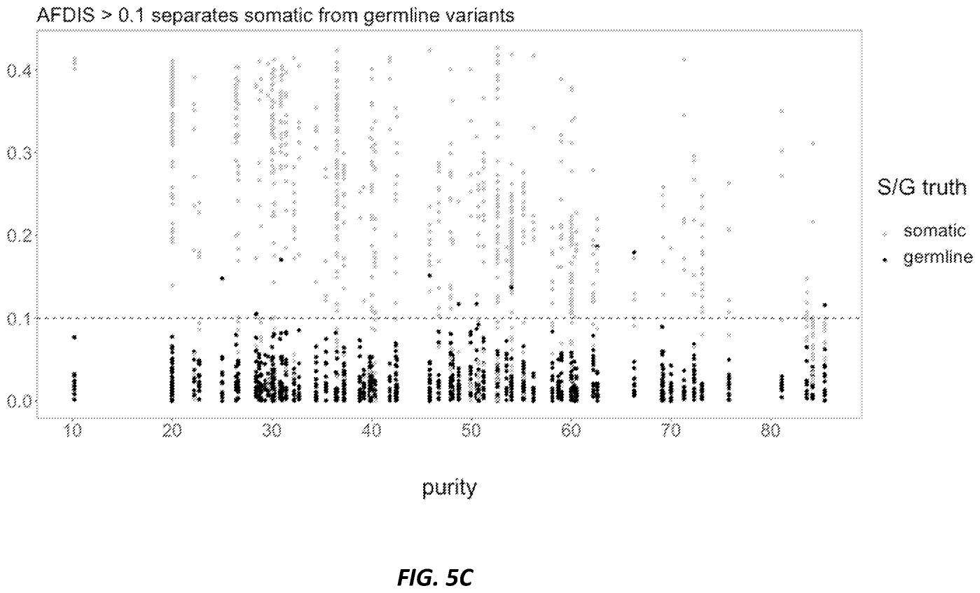

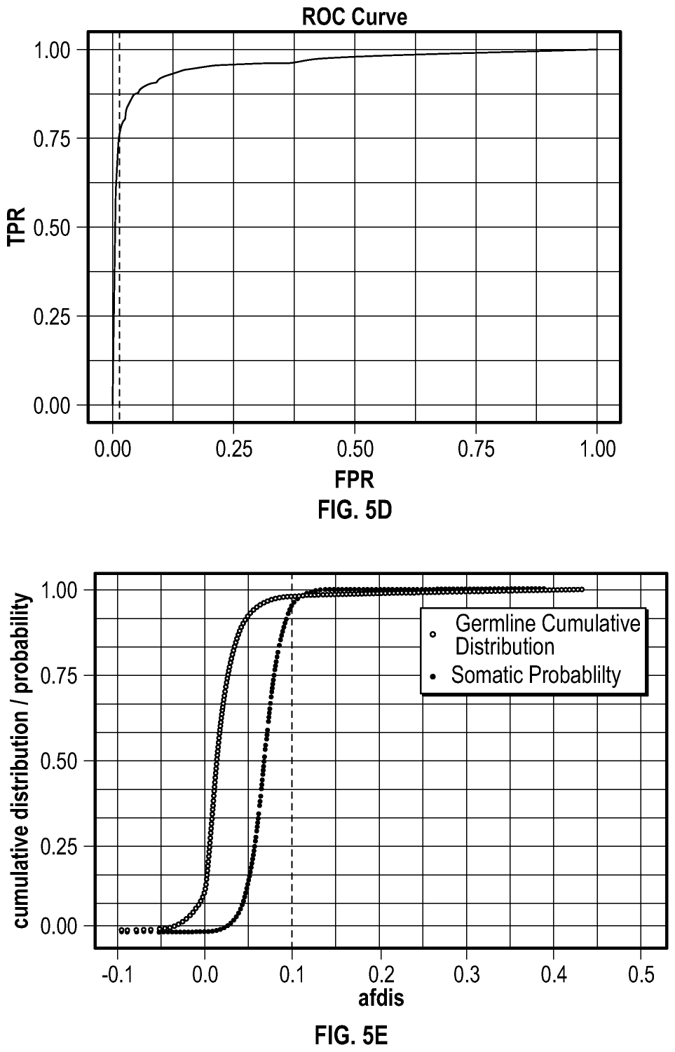

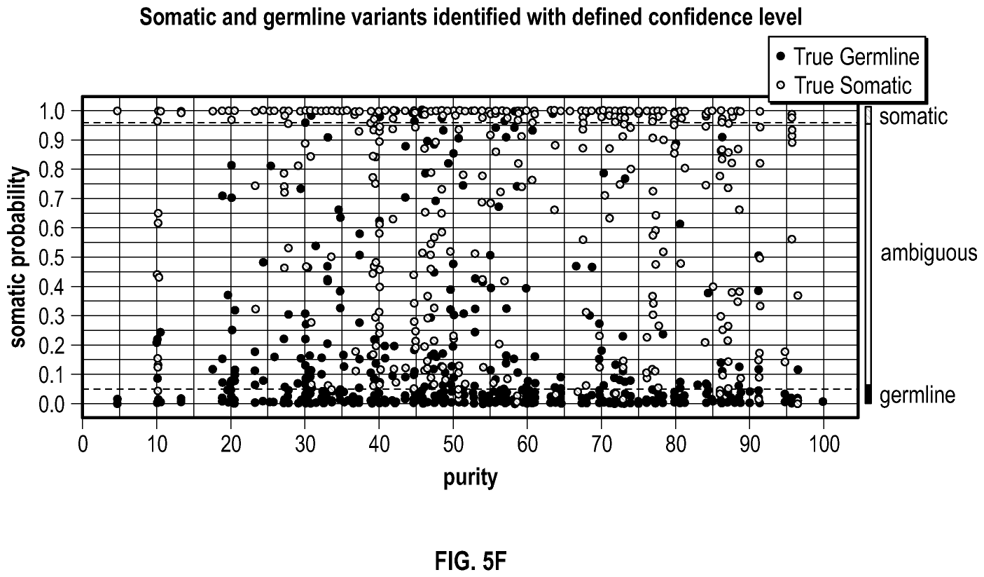

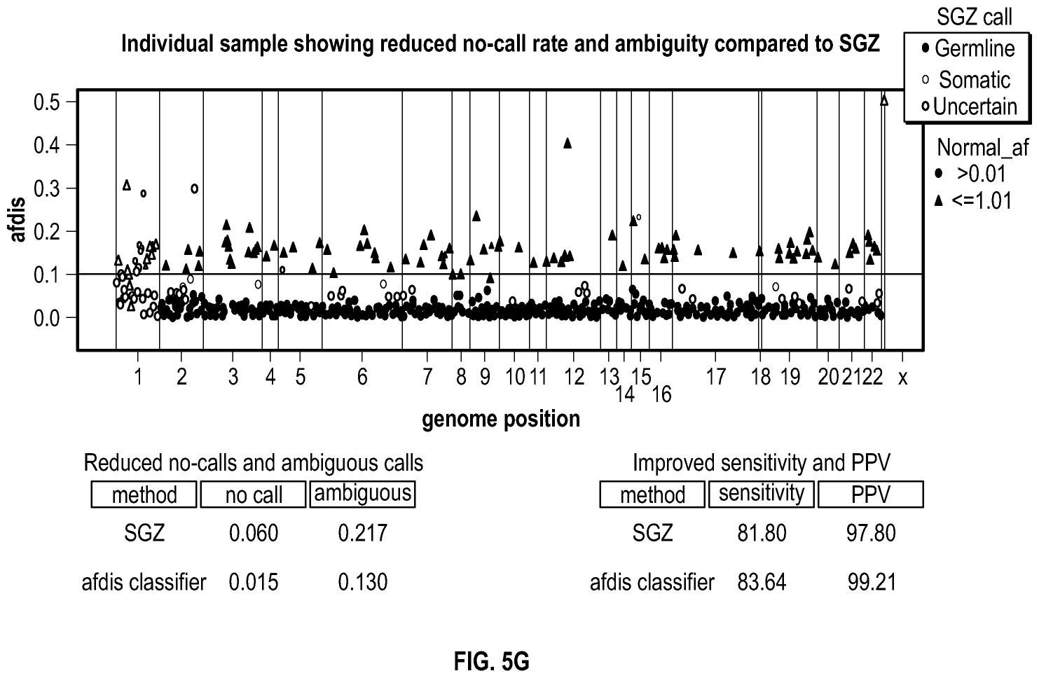

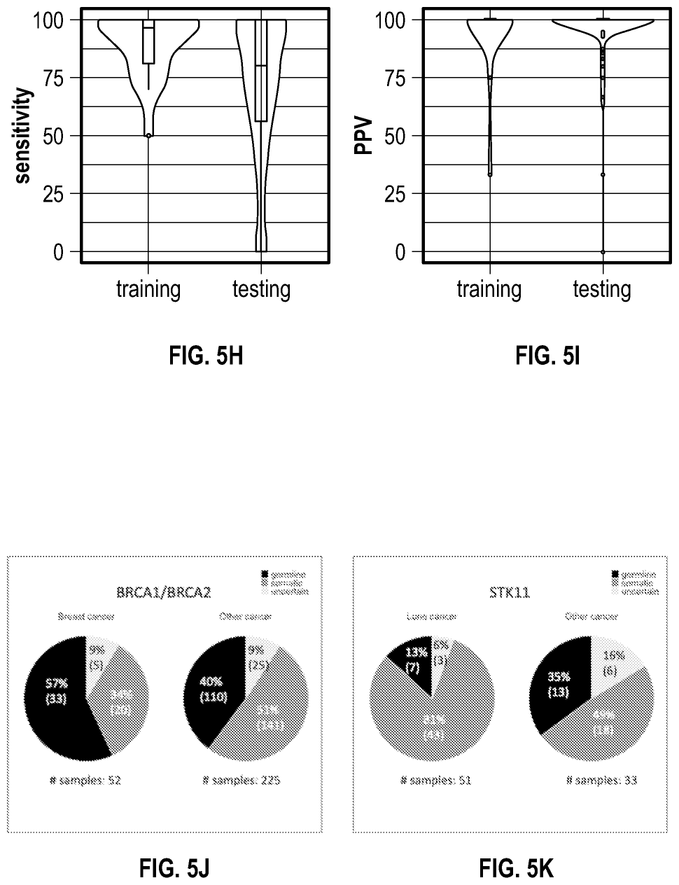

Previously described SGZ algorithms (see, e.g., Sun, et al. (2018), ibid.) can be used to determine the difference in expected variant allele frequency for somatic and germline variants (e.g., a mutation that replaces a C with a T) provided that the tumor fraction for the sample, allele count of the variant, and copy number of the genomic locus were determined, as shown in FIG. 5A. The expected variant allele frequency (VAF) for somatic and germline variants can be determined as follows:

$$A {F}_{somatic} = \frac{p V}{p C + 2 ( 1 - p )}$$

$$A {F}_{germline} = \frac{p V + 1 - p}{p C + 2 ( 1 - p )}$$

wherein p is the tumor purity, V is the variant allele count, and C is the copy number of the allele. For example, given a tumor purity (p) of the sample as 0.25, a variant allele count (V) of 3, and a copy number (C) of 4, if the variant is somatic the expected allele frequency is 0.3 and if germline the expected allele frequency is 0.6. See, for example, Sun, et al., A computational approach to distinguish somatic vs. germline origin of genomic alterations from deep sequencing of cancer specimens without a matched normal, PLoS Comput Biol., vol. 14, no. 2, p. e1005965 (2018).

This Example provides an alternative approach to the previously described SGZ algorithms, which does not require modeling the tumor purity, variant allele count, or copy number values. The allele frequency distance from the expected germline allele frequency (AFDIS) is determined as:

AFDIS=AF_{germline}−AFᵥₐᵣᵢₐₙₜ

AF_{germline} is the allele frequency of the sequence assuming the sequence is a definitive germline sequence, as defined by the allele frequency of the corresponding proxy sequences. AFᵥₐᵣᵢₐₙₜ is the observed allele frequency of the given sequence being characterized. To understand the allele frequency distance distribution for germline variants, genomic sequences from 3802 tumor samples were segmented based on copy number uniformity using the Circular Binary Segmentation algorithm described in Olshen, et al., Circular Binary Segmentation for the Analysis of Array-Based DNA Copy Number Data, Biostatistics vol. 5, no. 4, pp. 557-572 (October 2004). Approximately 2.1 million known germline variants (identified in the dbSNP and/or gnomAD database) from the 3802 samples were selected, and the allele frequency (based on sequencing) of each germline variant was compared to the median allele frequency of proxy sequences within the same segment to determine the allele frequency distance of each germline variant. The probability density of the ˜2.1 million germline variants from 3,802 samples is shown in FIG. 5B and select values are shown in Table 2. An empirical cumulative distribution function (ECDF) was built from this germline AFDIS distribution data, which can be used to evaluate the probability of a given AFDIS being derived from a germline variant.

TABLE 2

| AFDIS | ECDF_{AFDIS} |
|---|---|
| 0.0535 | 0.951 |
| 0.0665 | 0.976 |
| 0.0711 | 0.980 |
| 0.1 | 0.993 |

A threshold of 0.1 AFDIS, corresponding to a cumulative distribution of 0.993 based on the above mentioned ECDF, was empirically determined to be capable of separating somatic from germline variants effectively. As indicated in Table 2, AFDIS thresholds ranging from about 0.05 to 0.1 all provided good discrimination between somatic and germline variants. Nevertheless, as explained below, a trained statistical model was built to understand the probability of any given sequence being germline or somatic.

Allele frequency distance was then determined for 92 genotype-matched high purity/low purity tumor samples with known germline sequences, somatic sequences, and tumor purity. The low purity sample was used to establish ground truth for the somatic/germline status of selected sequences, as in general a low purity sample is considered to be a close approximation of a normal sample and allows for reliable determination of somatic versus germline status of variants within. FIG. 5C shows variant AFDIS for germline and somatic sequences from the 92 tumor samples, plotted against sample computational purity. Grey circles indicate ground truth somatic sequences and black circles indicate ground truth germline sequences.

### Example 2—Logistic Regression of Somatic Germline Status Based on AFDIS

Using the available data from 21 matched tumor/normal pairs (lung squamous cell carcinoma (n=5), ovary serous carcinoma (n=4), lung adenocarcinoma (n=3), breast invasive ductal carcinoma (n=2), anus carcinoma (n=1), bladder urothelial carcinoma (n=1), CRC (n=1), kidney clear cell carcinoma (n=1), ovary high grade serous carcinoma (n=1), skin sarcoma (n=1), uterus endometrial adenocarcinoma (n=1), a logistical regression model was generated. The matched tumor/normal pairs allowed for confident determination of somatic and germline sequences. FIG. 5D shows a receiver operating characteristic (ROC) curve for this approach, i.e., a graphical plot of the classification model's true positive (TP) and false positive (FP) performance in discriminating between somatic and germline variants. The “leave-one-out cross-validation” (LOOCV) results for the model indicated an accuracy of 0.97 (95% confidence interval=[0.95, 0.99]) and a Cohen's (unweighted) Kappa statistic of 0.93. The model was trained using the matched tumor/normal pair data to output a probability that a given sequence is a somatic sequence. For a known germline sequence in the training data, the probability of the sequence being somatic is 0. For a known somatic sequence in the training data, the probability of the sequence being somatic is 1. The logistic regression model was trained, using the training data set, according to a function of:

$${log}_{10} ( \frac{{p}_{somatic}}{1 - {p}_{somatic}} ) ∼ AFDIS$$

wherein p_{somatic} is the probability that a given variant is a somatic variant. See FIG. 5E. In this non-limiting example, sequences for which p_{somatic}>0.5 were called somatic and all others were called germline.

The AFDIS data calculated as discussed above for variants in a total of 188 tumor samples in three different testing sets were inputted into the trained model to determine the probability of each selected sequence being somatic or germline. Based on somatic variant probability, the variant sequence was labeled as somatic (if above the somatic probability threshold), germline (if below the germline probability threshold), or ambiguous (i.e., between the somatic probability threshold and the germline probability threshold). See FIG. 5F.

The results of classification by the AFDIS classifier for a set of 93 tumor samples with matched normal samples used in the validation of the prior SGZ method demonstrate an improvement over the prior SGZ methods, as shown in FIG. 5G. The genomic sequences of the 93 tumor samples was obtained using a hybrid-capture bait set different from those used in the training dataset, demonstrating that the AFDIS classifier is robust and applicable to genomic data collected in different ways. A non-limiting example of the variant-level performance (# true positives (True), # false positives (FP), and positive predictive value (PPV)) of the method is summarized in Table 3.

TABLE 3

| Dataset | # Samples | Classified | True | FP | Sensitivity | PPV |
|---|---|---|---|---|---|---|
| Training | 21 | 304 | 333 | 3 | 90.39 | 99.01 |
| Testing 1 | 6 | 30 | 32 | 2 | 87.50 | 93.33 |
| Testing 2 | 93 | 505 | 599 | 4 | 83.64 | 99.21 |

A non-limiting example of data for the sample-level sensitivity performance of the method is shown in FIG. 5H, and a non-limiting example of data for the positive predictive value (PPV) performance is shown in FIG. 5I. In the “violin plots” shown in FIG. 5H and FIG. 5I, the shape of the plot indicates the probability density of values on the vertical axis. The box-and-whisker plots nested inside the violin plots indicate the median, first and third quartile, minimum, maximum, and outlier values for the parameter plotted on the vertical axis. For the PPV plot of this example, the majority of samples had a PPV of 100%, therefore the median, maximum, and first and third quartile indicators are compressed.

Non-limiting examples of data for classification of variants in the BRCA1 and BRCA 2 genes is shown in FIG. 5J. Non-limiting examples of data for classification of variants in the STK11 gene is shown in FIG. 5K. As expected, BRCA1 and BRCA2 mutations were found to be enriched in germline origin variants among breast cancer compared to other cancer types (p=0.025 chi-squared test), and STK11 mutations were found to be enriched in somatic origin variants among lung cancer compared to other cancer types (p=0.0026 chi-squared test).

### Example 3—Logistic Regression of Somatic Germline Status Based on AFDIS

The disclosed methods for discriminating between somatic and germline variants are based on a comparison of allele frequency (AF) of the variant in question to the allele frequencies of known variants in close proximity to its genomic location. In some instances, as noted above, known germline variants in germline databases (e.g., public databases) can be used for comparison. If the AF of the variant in question is very similar to, or very different from, those of the known germline variants located in close proximity, one would conclude that the variant in question is very likely, or unlikely, to be germline, respectively.

In general, the AF of a given variant is mainly decided by its copy number as well as the tumor fraction of the sample. Tumor fraction is a constant for a particular sample, thus the AF of a given variant in a given sample is largely decided by its copy number. This means that, to infer somatic/germline status of a variant, AF can be compared to the AF of germline variants of the same copy number. Two non-limiting examples of implementing such comparisons are described below and in Example 4.

In one implementation, one calculates an “allele frequency distance” (AFDIS) that represents the distance between the AF of the variant in question and the median AF of germline variants located on the same copy number segment (e.g., located on the same physically continuous piece of genomic segment, or located on a discontinuous piece of genomic segment as long as the segment is present at the same copy number as the variant in question). Initially, AFDIS was calculated as:

AFDIS=|MAFᵥₐᵣᵢₐₙₜ−MAF_{segment}|

where MAF=minor allele frequency, i.e., the minor allele frequency for both the variant of interest and the median of the minor allele frequencies for the segment germline variants was used to calculate their absolute distance. A logistic regression model was then trained with a training dataset consisting of known somatic and germline variants to capture the relationship between “somatic probability” and AFDIS. The model was subsequently improved by using a distance with direction, i.e., redefining AFDIS as AFDIS=AF_{segment}−AFᵥₐᵣᵢₐₙₜ, where AF_{segment} is the median of the allele frequencies for the segment germline variants. In this equation, the sign of AFDIS accounts for somatic variants having a lower allele frequency compared to germline variants of the same copy number when there is normal tissue, cells, or cfDNA admixed in the sample. This is because sequencing reads originating from the normal part of the sample or from normal cells in the blood carry germline variants but not somatic variants. The logistic regression model is trained to recognize that negative AFDIS is associated with a low probability of the variant being somatic. The use of the directional AFDIS calculation improved the performance of the model for discriminating between somatic and germline variants.

The AFDIS-based approach has an advantage of simplicity and ease of calculation, and thus can be easily modified to include other considerations in a given implementation. Specifically, since AFDIS is the single predictive variable in the logistic regression model, one can easily adjust the AFDIS value to modify the outcome to account for other potential technical issues. For example, to account for increased uncertainty introduced by mild contamination of the nucleic acid sample, one can apply an adjustment to the AFDIS value according to the contamination level to move the AFDIS value into a range corresponding to more accurate classification of somatic/germline variants by the model. Similar adjustments can be made to account for additional uncertainties introduced by factors such as low read depth, noisy AF estimation, low segment germline SNP count, high variability in segment germline SNP AF, etc. The degree and manner of implementing these adjustments can be engineered and tuned using training datasets comprising known somatic and germline variants.

### Example 4—Germline Exclusion Based on the Probability Distribution of Germline Allele Frequencies

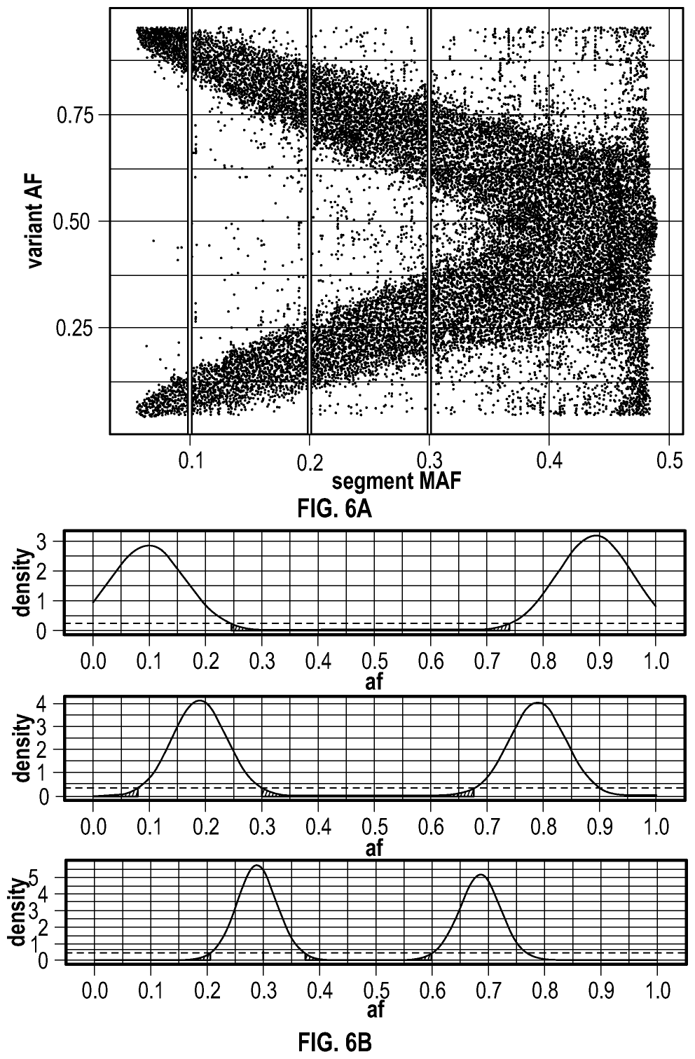

In this particular implementation, a large dataset of known germline variants was constructed, each with their own AF and the corresponding segment MAF, which is the median MAF of other known germline variants located in the same copy number segment FIG. 6A shows a plot of variant AF versus segment MAF. For an unknown variant to be classified, its AF and corresponding segment MAF are determined. To classify the unknown variant, data is taken from the known germline dataset which includes a subset of known germline variants having a segment MAF similar to that of the unknown variant (for example, one of the three density versus variant AF plots shown in FIG. 6B, which correspond to the variant allele frequency distributions at segment MAF near 0.1, 0.2, and 0.3, respectively, as shown in FIG. 6A). Using this data, one can establish a distribution of germline AF values for the given segment MAF (i.e., the given copy number, because segment MAF is intrinsically decided by the copy number of the segment). The AF of the unknown variant is compared to this germline AF distribution to infer the probability of the unknown variant being a germline variant. For example, an unknown variant having an AF of either 0.1 or 0.9, and a segment MAF of 0.1 is likely a germline variant, whereas an unknown variant having an AF of 0.4 and a segment MAF of 0.1 is likely a somatic variant.

### Example 5—Performance Verification

The disclosed methods provide exemplary techniques for selecting somatic variants from baseline tissue or liquid biopsy samples for plasma monitoring. Several additional measures have been devised to further enhance performance for this particular purpose, including: i) selection of well-behaved variants (e.g., by excluding variants located in genomic regions known to have or expected to have allele frequencies deviating from expected values (such as variants located in regions with repetitive sequences or in regions that share homology with other regions of the genome)) for constructing the logistic regression model, ii) incorporating prior knowledge of the likelihood of a variant being a germline, somatic, or clonal hematopoiesis of indeterminate potential (CHIP) variant based on historical data and public databases, and iii) taking into consideration the noise level of the variant call and its genomic context. These measures were found to enhance performance of somatic variant classification.

The ability of the disclosed AFDIS-based logistic regression models to distinguish somatic variants from germline variants in a sample was verified using, for example, data from matched tumor/normal pairs. The initial training and test datasets used for developing the logistic regression model and non-limiting examples of the resulting performance metrics (# false positives (FP), sensitivity, and positive predictive value (PPV)) for variant-level and sample-level performance are summarized in Table 4 and Table 5, respectively.

TABLE 4
Non-limiting example of variant-level performance data

| | True | Classified | | | |
|---|---|---|---|---|---|
| Dataset | Somatic | Somatic | FP | Sensitivity | PPV |
| Training set 1 | 469 | 422 | 4 | 89.13 | 99.05 |
| Training set 2 | 141 | 130 | 3 | 90.78 | 98.46 |
| Training set 3 | 848 | 610 | 7 | 71.11 | 98.85 |
| Training set 4 | 889 | 751 | 9 | 83.46 | 98.80 |
| Testing set | 1379 | 1233 | 6 | 88.98 | 99.51 |
| Total | 3726 | 3146 | 28 | 83.68 | 99.11 |

TABLE 5
Non-limiting example of sample-level performance data

| | Number of | | | | Percent |
|---|---|---|---|---|---|
| Dataset | Samples | FP = 0 | FP = 1 | FP > 1 | FP ≤ 1 |
| Training set 1 | 20 | 18 | 1 | 1 | 95 |
| Training set 2 | 6 | 4 | 2 | 0 | 100 |
| Training set 3 | 89 | 82 | 7 | 0 | 100 |
| Training set 4 | 93 | 85 | 7 | 1 | 98.9 |
| Testing set | 60 | 54 | 6 | 0 | 100 |
| Total | 268 | 243 | 23 | 2 | 99.3 |

The dataset used in a variant calling pipeline verification study included data from 86 matched tissue/peripheral blood mononuclear cell (PBMC) sample pairs. The variant-level and sample-level performance metrics are summarized in Table 6 and Table 7, respectively.

TABLE 6
Non-limiting example of variant-level performance

| | |
|---|---|
| Truth | 1769 |
| TP | 1432 |
| FP | 15 |
| Sensitivity | 80.95 |
| PPV | 98.96 |

TABLE 7
Non-limiting example of sample-level performance

| | Count | Percent |
|---|---|---|
| # Samples | 86 | 100.0% |
| FP = 0 | 74 | 86.0% |
| FP = 1 | 9 | 10.5% |
| FP = 2 | 3 | 3.5% |

The dataset used in additional variant calling pipeline verification studies included data from 746 matched tissue/peripheral blood mononuclear cell (PBMC) sample pairs. The variant-level and sample-level performance metrics are summarized in Table 8 and Table 9, respectively.

TABLE 8
Non-limiting example of variant-level performance

| | |
|---|---|
| Truth | 6331 |
| TP | 4255 |
| FP | 112 |
| Sensitivity | 67.21 |
| PPV | 97.44 |

TABLE 9
Non-limiting example of sample-level performance

| | Count | Percent |
|---|---|---|
| VarCount > 0 | 727 | 97.45 |
| FP = 0 | 633 | 87.07 |
| FP = 1 | 80 | 11.00 |
| FP = 2 | 11 | 1.51 |
| FP > 2 | 3 | 0.41 |

It will be appreciated that the methods and systems described above are set forth by way of example and not of limitation. Numerous variations, additions, omissions, and other modifications will be apparent to one of ordinary skill in the art. In addition, the order or presentation of method steps in the description and drawings above is not intended to require this order of performing the recited steps unless a particular order is expressly required or otherwise clear from the context.

The method steps of the invention(s) described herein are intended to include any suitable method of causing one or more other parties or entities to perform the steps, unless a different meaning is expressly provided or otherwise clear from the context. In some aspects, such parties or entities need not be under the direction or control of any other party or entity, and need not be located within a particular jurisdiction. Thus, for example, a description or recitation of “adding a first number to a second number” includes causing one or more parties or entities to add the two numbers together. For example, if person X engages in an arm's length transaction with person Y to add the two numbers, and person Y indeed adds the two numbers, then both persons X and Y perform the step as recited: person Y by virtue of the fact that he actually added the numbers, and person X by virtue of the fact that he caused person Y to add the numbers. Furthermore, if person X is located within the United States and person Y is located outside the United States, then the method is performed in the United States by virtue of person X's participation in causing the step to be performed.

The terminology used in the description of the various described embodiments herein is for the purpose of describing particular embodiments only and is not intended to be limiting. As used in the description of the various described embodiments and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “includes,” “including,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof.

The disclosures of all publications, patents, and patent applications referred to herein are each hereby incorporated by reference in their entireties. To the extent that any reference incorporated by reference conflicts with the instant disclosure, the instant disclosure shall control.

While particular embodiments of the present invention have been shown and described, it will be apparent to those skilled in the art that various changes and modifications in form and details may be made therein without departing from the spirit and scope of the invention as defined by the following claims. The claims that follow are intended to include all such variations and modifications that might fall within their scope, and should be interpreted in the broadest sense allowable by law.

What is claimed is:

1. A method of identifying a genomic sequence of interest as germline or somatic, the method comprising:
providing a plurality of nucleic acid molecules obtained from a sample from a subject, wherein the plurality of nucleic acid molecules comprises a mixture of tumor nucleic acid molecules and non-tumor nucleic acid molecules;
optionally, ligating one or more adapters onto one or more nucleic acid molecules from the plurality of nucleic acid molecules;
amplifying nucleic acid molecules from the plurality of nucleic acid molecules;
capturing nucleic acid molecules from the amplified nucleic acid molecules, wherein the captured nucleic acid molecules are captured from the amplified nucleic acid molecules by hybridization to one or more bait molecules;
sequencing, by a sequencer, the captured nucleic acid molecules to obtain a plurality of sequence reads corresponding to one or more genomic loci;
selecting, by one or more processors, a genomic sequence of interest at a genomic locus from the one or more genomic loci;
selecting, by the one or more processors, one or more proxy genomic sequences for the genomic sequence of interest;
determining, by the one or more processors, an allele frequency distance using an observed allele frequency of the genomic sequence of interest and a summary statistic or distribution indicative of observed allele frequencies of the one or more proxy genomic sequences; and
identifying, by the one or more processors, the genomic sequence of interest as germline or somatic using the allele frequency distance.

2. The method of claim 1, wherein the subject is a cancer patient.

3. The method of claim 1 or claim 2, wherein the sample comprises a tissue biopsy sample, a liquid biopsy sample, a circulating tumor cell (CTC) sample, a cell-free DNA (cfDNA) sample, or a normal control.

4. The method of claim 3, wherein the sample is a liquid biopsy sample and comprises blood, plasma, cerebrospinal fluid, sputum, stool, urine, or saliva.

5. The method of any one of claims 1 to 3, wherein the tumor nucleic acid molecules are derived from a tumor portion of a heterogeneous tissue biopsy sample, and the non-tumor nucleic acid molecules are derived from a normal portion of the heterogeneous tissue biopsy sample.

6. The method of any one of claims 1 to 3, wherein the tumor nucleic acid molecules are derived from a circulating tumor DNA (ctDNA) fraction of a cell-free DNA sample, and the non-tumor nucleic acid molecules are derived from a non-tumor fraction of the cell-free DNA sample.

7. The method of any one of claims 1 to 6, wherein the one or more adapters comprise amplification primers or sequencing adapters.

8. The method of any one of claims 1 to 7, wherein the one or more bait molecules comprise one or more nucleic acid molecules, each comprising a region that is complementary to a region of a captured nucleic acid molecule.

9. The method of any one of claims 1 to 8, wherein amplifying nucleic acid molecules comprises performing a polymerase chain reaction (PCR) or isothermal amplification technique.

10. The method of any one of claims 1 to 9, wherein the sequencing comprises use of a next generation sequencing (NGS) technique.

11. The method of any one of claims 1 to 10, wherein the sequencer comprises a next generation sequencer.

12. The method of any one of claims 1 to 11, wherein the one or more proxy genomic sequences are located within a defined segment of the subject's genomic sequence, and the selected genomic sequence of interest is located within the same defined segment.

13. The method of claim 12, wherein the subject's genomic sequence is segmented into a plurality of segments based on copy number uniformity within each segment.

14. The method of any one of claims 1 to 13, wherein the summary statistic is a mean allele frequency or a median allele frequency.

15. The method of any one of claims 1 to 14, wherein the allele frequency distance is determined using the observed allele frequency of the genomic sequence of interest and the distribution indicative of the observed frequencies of a plurality of proxy genomic sequences, and wherein the genomic sequence of interest is identified as germline or somatic based on a probability that the observed allele frequency of the genomic sequence of interest fits within or does not fit within the distribution.

16. A method of identifying a genomic sequence of interest as germline or somatic, the method comprising:
selecting, by one or more processors, a genomic sequence of interest at a genomic locus from within a patient genomic sequence obtained for a patient sample comprising a mixture of tumor nucleic acid molecules and non-tumor nucleic acid molecules;
selecting, by the one or more processors, one or more proxy genomic sequences for the genomic sequence of interest;
determining, by the one or more processors, an allele frequency distance using an observed allele frequency of the genomic sequence of interest and a summary statistic or distribution indicative of observed allele frequencies of the one or more proxy genomic sequences; and
identifying, by the one or more processors, the genomic sequence of interest as germline or somatic using the allele frequency distance.

17. The method of claim 16, comprising sequencing, by a sequencer, the tumor nucleic acid molecules and the non-tumor nucleic acid molecules from the patient sample to determine the patient genomic sequence.

18. The method of claim 17, wherein the patient genomic sequence is obtained using a next generation sequencing technique.

19. The method of claim 17, wherein the sequencer is a next generation sequencer.

20. The method of any one of claims 16 to 19, wherein the one or more proxy genomic sequences are located within a defined segment of the patient genomic sequence, and the selected genomic sequence of interest is located within the same defined segment.

21. The method of claim 20, wherein the patient genomic sequence is segmented into a plurality of segments based on copy number uniformity within each segment.

22. The method of claim 20 or 21, comprising segmenting the patient genomic sequence into a plurality of segments.

23. The method of any one of claims 16 to 22, wherein the summary statistic is a mean allele frequency or a median allele frequency.

24. The method of any one of claims 16 to 23, wherein the allele frequency distance is determined using the observed allele frequency of the genomic sequence of interest and the distribution indicative of the observed frequencies of a plurality of proxy genomic sequences, and wherein the genomic sequence of interest is identified as germline or somatic based on a probability that the observed allele frequency of the genomic sequence of interest fits within or does not fit within the distribution.

25. The method of any one of claims 16 to 24, wherein the tumor nucleic acid molecules and the non-tumor nucleic acid molecules comprise DNA molecules.

26. The method of any one of claims 16 to 25, wherein the tumor nucleic acid molecules and the non-tumor nucleic acid molecules comprise RNA molecules.

27. The method of any one of claims 16 to 26, wherein the patient genomic sequence is determined using targeted sequencing.

28. The method of claim 27, wherein the targeted sequencing comprises targeted sequencing of one or more genes associated with cancer, or a portion thereof.

29. The method of claim 27 or claim 28, wherein the targeted sequencing comprises targeted sequencing of one or more exon regions.

30. A method of identifying a genomic sequence of interest as germline or somatic, the method comprising:
identifying, by one or more processors, a genomic sequence of interest in a patient sample at a genomic locus;
identifying, by the one or more processors, one or more proxy genomic sequences for the sequence of interest;
comparing, by the one or more processors, an observed frequency of the genomic sequence of interest to a centrality measure of observed frequencies of the one or more proxy genomic sequences; and
identifying, by the one or more processors, the genomic sequence of interest as germline or somatic based on the comparison.

31. The method of claim 30, further comprising identifying, by the one or more processors, a segment of a patient's genome in which the genomic locus is included.

32. The method of claim 31, wherein identifying, by the one or more processors, the segment includes performing a segmentation procedure on a continuous portion of the patient's genome.

33. The method of claim 32, wherein the portion of the patient's genome is large enough to identify three distinct segments.

34. The method of claim 31, wherein the proxy is identified, by the one or more processors, to be located within the same segment as the genomic locus.

35. The method of claim 32, wherein the segmentation procedure identifies, by the one or more processors, segments according to whether a genomic parameter is equal across the entirety of each individual segment.

36. The method of claim 35, wherein the genomic parameter is copy number.

37. The method of any one of claims 16 to 36, wherein identifying, by the one or more processors, the genomic sequence of interest as germline or somatic comprises:
inputting an allele frequency distance into a trained statistical model; and
outputting, from the trained statistical model, a value indicative of a likelihood that the genomic sequence of interest is germline or a value indicative of a likelihood that the genomic sequence of interest is somatic.

38. The method of any one of claims 16 to 37, wherein the allele frequency distance is adjusted to correct for a contamination level in the patient sample, a low sequencing read depth, a noisy estimation of allele frequencies, a low segment germline single nucleotide polymorphism (SNP) count, or high variability in segment germline SNP allele frequency.

39. The method of claim 37 or claim 38, wherein the trained statistical model comprises a function that associates the allele frequency distance with the value indicative of a likelihood that the genomic sequence of interest is germline or the value indicative of a likelihood that the genomic sequence of interest is somatic.

40. The method of any one of claims 37 to 39, wherein the trained statistical model is a logistic regression model.

41. The method of any one of claims 37 to 40, further comprising training the statistical model using data for tumor samples with known germline sequences.

42. The method of any one of claims 37 to 41, further comprising training the statistical model using data for tumor samples with known germline sequences and known somatic sequences.

43. The method of any one of claims 37 to 40, wherein the trained statistical model is trained using data for tumor samples with known germline sequences.

44. The method of claim 43, wherein the trained statistical model is trained using data for tumor samples with known germline sequences and known somatic sequences.

45. The method of any one of claim 37 to 44, further comprising training the statistical model using data for variant allele frequencies that excludes variants located in genomic regions known to have allele frequencies that deviate from expected values.

46. The method of any one of claims 37 to 44, wherein the trained statistical model is trained using data for variant allele frequencies that excludes variants located in genomic regions known to have allele frequencies that deviate from expected values.

47. The method of any one of claims 37 to 46, further comprising training the statistical model using data that incorporates prior knowledge of the likelihood of a variant being a germline, a somatic variant, or a clonal hematopoiesis of indeterminate potential (CHIP) variant based on historical data or databases.

48. The method of any one of claims 37 to 46, wherein the trained statistical model is trained using data that incorporates prior knowledge of the likelihood of a variant being a germline, a somatic variant, or a clonal hematopoiesis of indeterminate potential (CHIP) variant based on historical data or databases.

49. The method of any one of claims 37 to 48, further comprising training the statistical model using data that accounts for a noise level for a given variant call and its genomic context.

50. The method of any one of claims 37 to 48, wherein the trained statistical model is trained using data that accounts for a noise level for a given variant call and its genomic context.

51. The method of any one of claims 16 to 50, wherein the one or more proxy genomic sequences include a single nucleotide polymorphism (SNP).

52. The method of any one of claims 16 to 51, wherein the one or more proxy genomic sequences include an allele.

53. The method of any one of claims 16 to 52, wherein the genomic sequence of interest includes a genomic variant.

54. The method of any one of claims 16 to 53, further comprising generating, by the one or more processors, a report indicating the genomic sequence of interest as germline or somatic.

55. The method of claim 54, comprising transmitting the report to a healthcare provider.

56. The method of claim 54 or claim 55, wherein the report is transmitted via a computer network or a peer-to-peer connection.

57. The method of any one of claims 16 to 56, wherein the patient sample is derived from a tissue biopsy comprising tumor tissue and non-tumor tissue.

58. The method of claim 57, wherein the tissue biopsy is a solid tissue biopsy or a liquid biopsy.

59. The method of claim 58, wherein the tissue biopsy is a liquid biopsy comprising blood, plasma, cerebrospinal fluid, sputum, stool, urine, or saliva.

60. The method of any one of claims 16 to 59, wherein the patient sample comprises cell-free DNA (cfDNA) obtained from the subject.

61. The method of any one of claims 16 to 60, wherein the patient sample comprises circulating tumor DNA (ctDNA) obtained from the subject.

62. A method of treating cancer in a patient, comprising:
identifying, by the one or more processors, one or more genomic sequences of interest as somatic using the method of any one of claims 16 to 61;
selecting a cancer treatment modality based on the one or more identified somatic sequences; and
treating the cancer using the selected cancer treatment modality.

63. The method of claim 62, wherein the one or more identified somatic sequences are associated with successful cancer treatment using the selected treatment modality.

64. The method of claim 62, comprising:
determining, by the one or more processors a microsatellite instability status of the cancer using the one or more identified somatic sequences; and
selecting the cancer treatment modality based on the microsatellite instability status of the cancer.

65. The method of claim 62, comprising:
determining, by the one or more processors, a tumor mutational burden for the cancer using the one or more identified somatic sequences; and
selecting the cancer treatment modality based on the tumor mutational burden being above a predetermined tumor mutational burden threshold.

66. The method of claim 64 or claim 65, wherein the cancer treatment modality comprises administration of an effective amount of one or more anti-cancer agents to the patient if the tumor mutational burden is above a predetermined threshold.

67. The method of claim 66, wherein the one or more anti-cancer agents comprises an immuno-oncology agent.

68. The method of claim 67, wherein the immuno-oncology agent is an immune checkpoint inhibitor.

69. A method of monitoring cancer progression or recurrence in a patient, comprising:
identifying, by the one or more processors, one or more genomic sequences of interest as somatic using the method of any one of claims 16 to 67, wherein the patient sample is obtained from a patient having cancer; and
detecting, by the one or more processors, the presence or absence of the one or more genomic sequences of interest identified as somatic within a second patient sample obtained from patient after the cancer has been treated.

70. The method of claim 69, comprising obtaining the second patient sample from the patient.

71. The method of claim 69 or claim 70, comprising treating the cancer in the patient after the first patient sample is obtained from the patient and before the second patient sample is obtained from the patient.

72. The method of any one of claims 69 to 71, wherein the second patient sample comprises cell-free DNA.

73. The method of any one of claims 69 to 72, wherein detecting the presence or absence of the one or more genomic sequences of interest identified as somatic within the second patient sample comprises sequencing nucleic acid molecules in the second patient sample.

74. A method of selecting a neoantigen for a cancer vaccine personalized for a subject having cancer, comprising:
identifying, by the one or more processors, one or more genomic sequences of interest as somatic using the method of any one of claims 16 to 67, wherein the one or more genomic sequences of interest identified as somatic is located within an exon region of a gene; and
selecting, by the one or more processors, from the one or more genomic sequences of interest identified as somatic, a genomic sequence that encodes a neoantigen suitable as a cancer vaccine for the subject.

75. The method of claim 74, further comprising making a vaccine comprising the neoantigen.

76. A non-transitory computer-readable storage medium storing one or more programs, the one or more programs comprising instructions, which when executed by one or more processors of an electronic device, cause the electronic device to:
select a genomic sequence of interest at a genomic locus from within a patient genomic sequence obtained for a patient sample comprising a mixture of tumor nucleic acid molecules and non-tumor nucleic acid molecules;
select one or more proxy genomic sequences for the genomic sequence of interest;
determine an allele frequency distance using an observed allele frequency of the genomic sequence of interest and a summary statistic or distribution indicative of observed allele frequencies of the one or more proxy genomic sequences; and
identify the genomic sequence of interest as germline or somatic using the allele frequency distance.

77. The non-transitory computer-readable storage medium of claim 76, wherein the one or more proxy genomic sequences are located within a defined segment of the patient genomic sequence, and the selected genomic sequence of interest is located within the same defined segment.

78. The non-transitory computer-readable storage medium of claim 77, wherein the patient genomic sequence is segmented into a plurality of segments based on copy number uniformity within each segment.

79. The non-transitory computer-readable storage medium of claim 77 or claim 78, wherein the one or more programs further comprise instructions, which when executed by the one or more processors of the electronic device, cause the electronic device to segment the patient genomic sequence into a plurality of segments.

80. The non-transitory computer-readable storage medium of any one of claims 76 to 79, wherein the summary statistic is a mean allele frequency or a median allele frequency.

81. The non-transitory computer-readable storage medium of any one of claims 76 to 80, wherein the allele frequency distance is determined using the observed allele frequency of the genomic sequence of interest and the distribution indicative of the observed frequencies of a plurality of proxy genomic sequences, and wherein the genomic sequence of interest is identified as germline or somatic based on a probability that the observed allele frequency of the genomic sequence of interest fits within or does not fit within the distribution.

82. The non-transitory computer-readable storage medium of any one of claims 76 to 81, wherein the tumor nucleic acid molecules and the non-tumor nucleic acid molecules comprise DNA molecules.

83. The non-transitory computer-readable storage medium of any one of claims 76 to 82, wherein the tumor nucleic acid molecules and the non-tumor nucleic acid molecules comprise RNA molecules.

84. The non-transitory computer-readable storage medium of any one of claims 76 to 83, wherein the patient genomic sequence is determined using targeted sequencing.

85. The non-transitory computer-readable storage medium of any one of claims 76 to 84, wherein the patient genomic sequence is determined using next generation sequencing.

86. The non-transitory computer-readable storage medium of claim 84 or claim 85, wherein the targeted sequencing comprises targeted sequencing of one or more genes associated with cancer, or a portion thereof.

87. The non-transitory computer-readable storage medium of any one of claim 84 to 86, wherein the targeted sequencing comprises targeted sequencing of one or more exon regions.

88. A non-transitory computer-readable storage medium storing one or more programs, the one or more programs comprising instructions, which when executed by one or more processors of an electronic device, cause the electronic device to:
identify a genomic sequence of interest in a patient sample at a genomic locus;
identify one or more proxy genomic sequences for the sequence of interest;
identify an observed frequency of the sequence of interest to a centrality measure of observed frequencies of the one or more proxy genomic sequences; and
based on the comparison, characterize the genomic sequence of interest as either germline or somatic.

89. The non-transitory computer readable storage medium of claim 88, wherein the one or more programs further comprise instructions, which when executed by the one or more processors of the electronic device, cause the electronic device to identify a segment of a patient's genome in which the genomic locus is included.

90. The non-transitory computer readable storage medium of claim 88, wherein identifying the segment includes performing a segmentation procedure on a continuous portion of the patient's genome.

91. The non-transitory computer readable storage medium of claim 90, wherein the portion of the patient's genome is large enough to identify three distinct segments.

92. The non-transitory computer readable storage medium of any one of claims 88 to 91, wherein the one or more proxy genomic sequences are identified to be located on the same segment as the genomic locus.

93. The non-transitory computer readable storage medium of any one of claims 90 to 92, wherein the segmentation procedure identifies segments according to whether a genomic parameter is equal across the entirety of each individual segment.

94. The non-transitory computer readable storage medium of claim 93, wherein the genomic parameter is copy number.

95. The non-transitory computer-readable storage medium of any one of claims 76 to 94, wherein the one or more programs further comprise instructions, which when executed by one or more processors of the electronic device, cause the electronic device to receive sequencing data associated with the patient genomic sequence.

96. The non-transitory computer-readable storage medium of claim 95, wherein the one or more programs further comprise instructions, which when executed by one or more processors of the electronic device, cause the electronic device to assemble the patient genomic sequence using the sequencing data.

97. The non-transitory computer-readable storage medium of claim 95 or claim 96, wherein the one or more programs further comprise instructions, which when executed by one or more processors of the electronic device, cause the electronic device to operate a sequencer to sequence nucleic acid molecules derived from the patient sample, thereby obtaining the sequencing data.

98. The non-transitory computer-readable storage medium of any one of claims 76 to 97, wherein the one or more programs further comprise instructions, which when executed by the one or more processors of the electronic device, cause the electronic device to generate a report indicating the genomic sequence of interest as either germline or somatic.

99. The non-transitory computer-readable storage medium of any one of claims 76 to 98, wherein the one or more programs further comprise instructions, which when executed by the one or more processors of the electronic device, cause the electronic device to transmit the report using a computer network.

100. The non-transitory computer readable storage medium of any one of claims 76 to 99, wherein the electronic device comprises a display, and the one or more programs further comprise instructions, which when executed by the one or more processors of the electronic device, cause the electronic device to display the report.

101. The non-transitory computer readable storage medium of any one of claims 76 to 100, wherein the one or more proxy genomic sequences includes a single nucleotide polymorphism (SNP).

102. The non-transitory computer readable storage medium of any one of claims 76 to 101, wherein the one or more proxy genomic sequences includes an allele.

103. The non-transitory computer readable storage medium of any one of claims 76 to 102, wherein the genomic sequence of interest includes a genomic variant.

104. An electronic device, comprising:
one or more processors; and
a memory storing one or more programs configured to be executed by the one or more processors, the one or more programs including instructions for:
selecting a genomic sequence of interest at a genomic locus from within a patient genomic sequence obtained for a patient sample comprising a mixture of tumor nucleic acid molecules and non-tumor nucleic acid molecules;
selecting one or more proxy genomic sequences for the genomic sequence of interest;
determining an allele frequency distance using an observed allele frequency of the genomic sequence of interest and a summary statistic or distribution indicative of observed allele frequencies of the one or more proxy genomic sequences; and
identifying the genomic sequence of interest as germline or somatic using the allele frequency distance.

105. The electronic device of claim 104, wherein the one or more proxy genomic sequences are located within a defined segment of the patient genomic sequence, and the selected genomic sequence of interest is located within the same defined segment.

106. The electronic device of claim 105, wherein the patient genomic sequence is segmented into a plurality of segments based on copy number uniformity within each segment.

107. The electronic device of any one of claims 104 to 106, wherein the one or more programs further include instructions for segmenting the patient genomic sequence into a plurality of segments.

108. The electronic device of any one of claims 104 to 107, wherein the summary statistic is a mean allele frequency or a median allele frequency.

109. The electronic device of any one of claims 104 to 108, wherein the allele frequency distance is determined using the observed allele frequency of the genomic sequence of interest and the distribution indicative of the observed frequencies of a plurality of proxy genomic sequences, and wherein the genomic sequence of interest is identified as germline or somatic based on a probability that the observed allele frequency of the genomic sequence of interest fits within or does not fit within the distribution.

110. The electronic device of any one of claims 104 to 109, wherein the tumor nucleic acid molecules and the non-tumor nucleic acid molecules comprise DNA molecules.

111. The electronic device of any one of claims 104 to 110, wherein the tumor nucleic acid molecules and the non-tumor nucleic acid molecules comprise RNA molecules.

112. The electronic device of any one of claims 104 to 111, wherein the patient genomic sequence is determined using next generation sequencing.

113. The electronic device of any one of claims 104 to 112, wherein the patient genomic sequence is determined using targeted sequencing.

114. The electronic device of claim 113, wherein the targeted sequencing comprises targeted sequencing of one or more genes associated with cancer, or a portion thereof.

115. The electronic device of claim 113 or claim 114, wherein the targeted sequencing comprises targeted sequencing of one or more exon regions.

116. An electronic device, comprising:
one or more processors; and
a memory storing one or more programs configured to be executed by the one or more processors, the one or more programs including instructions for:
identifying a genomic sequence of interest in a patient sample at a genomic locus;
identifying one or more proxy genomic sequences for the sequence of interest;
comparing an observed frequency of the genomic sequence of interest to a centrality measure of observed frequencies of the one or more proxy genomic sequences; and
identifying the genomic sequence of interest as germline or somatic based on the comparison.

117. The electronic device of claim 116, wherein the one or more programs further include instructions for identifying a segment of a patient's genome in which the genomic locus is included.

118. The electronic device of claim 117, wherein identifying the segment includes performing a segmentation procedure on a continuous portion of the patient's genome.

119. The electronic device of claim 118, wherein the portion of the patient's genome is large enough to identify three distinct segments.

120. The electronic device of any one of claims 117 to 119, wherein the one or more proxy genomic sequence are identified to be located within the same segment as the genomic locus.

121. The electronic device of any one of claims 118 to 120, wherein the segmentation procedure identifies segments according to whether a genomic parameter is equal across the entirety of each individual segment.

122. The electronic device of claim 121, wherein the genomic parameter is copy number.

123. The electronic device of any one of claims 104 to 122, wherein the one or more programs further comprise instructions for receiving sequencing data associated with the patient genomic sequence.

124. The electronic device of claim 123, wherein the one or more programs further comprise instructions for assembling the patient genomic sequence using the sequencing data.

125. The electronic device of claim 123 or claim 124, wherein the one or more programs further comprise instructions for causing a sequencer to sequence nucleic acid molecules derived from the patient sample, thereby obtaining the sequencing data.

126. The electronic device of any one of claims 104 to 125, wherein the one or more proxy genomic sequences includes a single nucleotide polymorphism (SNP).

127. The electronic device of any one of claims 104 to 126, wherein the one or more proxy genomic sequences includes an allele.

128. The electronic device of any one of claims 104 to 127, wherein the genomic sequence of interest includes a genomic variant.

129. The electronic device of any one of claims 104 to 128, wherein the one or more programs further include instructions for generating a report indicating the genomic sequence of interest as either germline or somatic.

130. The electronic device of claim 129, wherein the one or more programs further include instructions for transmitting the report via a computer network or peer-to-peer connection.

131. The electronic device of claim 129 or 130, wherein the device further comprises a display and the one or more programs further include instructions for displaying the report.

132. The electronic device of any one of claims 104 to 131, wherein the patient sample is derived from a tissue biopsy comprising tumor tissue and non-tumor tissue.

133. The electronic device of claim 132, wherein the tissue biopsy is a solid tissue biopsy or a liquid biopsy.

134. The electronic device of claim 133, wherein the tissue biopsy is a liquid biopsy comprising blood, plasma, cerebrospinal fluid, sputum, stool, urine, or saliva.

135. The electronic device of any one of claims 104 to 134, wherein the patient sample comprises cell-free DNA (cfDNA) obtained from the subject.

136. The electronic device of any one of claims 104 to 135, wherein the patient sample comprises circulating tumor DNA (ctDNA) obtained from the subject.

137. A system, comprising the electronic device of any one of claims 104 to 136 and a sequencer configured to sequence nucleic acid molecules derived from the patient sample.

138. The system of claim 137, wherein the sequencer is a next generation sequencer.

139. A method of identifying a genomic sequence of interest as germline or somatic, the method comprising:
identifying, by one or more processors, a genomic sequence of interest in a patient sample at a genomic locus;
identifying, by the one or more processors, a proxy genomic sequence for the genomic sequence of interest;
comparing, by the one or more processors, an observed allele fraction of the genomic sequence of interest to an observed allele fraction of the proxy genomic sequence; and
identifying, by the one or more processors, the genomic sequence of interest as germline or somatic based on the comparison.

140. The method of claim 139, wherein the proxy genomic sequence has the same copy number as the genomic sequence of interest.

141. The method of claim 139 or claim 140, wherein identifying, by the one or more processors, the genomic sequence of interest as germline or somatic comprises:
inputting an allele frequency distance into a trained statistical model; and
outputting, from the trained statistical model, a value indicative of a likelihood that the genomic sequence of interest is germline or a value indicative of a likelihood that the genomic sequence of interest is somatic.

142. The method of any one of claims 139 to 141, wherein the allele fraction of the genomic sequence and the allele fraction of the proxy genomic sequence are determined using a next generation sequencing technique.

143. The method of claim 142, wherein the allele fraction of the genomic sequence and the allele fraction of the proxy genomic sequence are determined using a microarray technique.

144. The method of any one of claims 139 to 143, wherein the patient sample comprises a solid tissue biopsy or a liquid biopsy.

145. The method of claim 144, wherein the patient sample is a liquid biopsy comprising blood, plasma, cerebrospinal fluid, sputum, stool, urine, or saliva.

146. The method of any one of claims 139 to 145, wherein the patient sample comprises cell-free DNA (cfDNA) obtained from the subject.

147. The method of any one of claims 139 to 146, wherein the patient sample comprises circulating tumor DNA (ctDNA) obtained from the subject.

148. The method of any one of claims 139 to 147, wherein the patient is a cancer patient.
